(19) United States
(12) Patent Application Publication
(10) Pub. No.: US 2025/0146970 A1
(43) Pub. Date: May 8, 2025

(54) Transition Metal Carbides And/Or Nitrides (Mxenes) For Electrochemical Sensing

(71) Applicants: Drexel University, Philadelphia, PA (US); University of Brighton, Brighton (GB); Rayner IOL Ltd, West Sussex (GB)

(72) Inventors: Maria Natalia Noriega Pedraza, Brighton (GB); Yury Gogotsi, Ivyland, PA (US); Bhavik Anil Patel, West Sussex (GB); Susan Rachel Sandeman, Brighton (GB); Marcus K. Dymond, Brighton (GB); Joseph Lacey, Philadelphia, PA (US)

(21) Appl. No.: 18/893,218

(22) Filed: Sep. 23, 2024

Related U.S. Application Data
(60) Provisional application No. 63/584,555, filed on Sep. 22, 2023.

(51) Int. Cl. G01N 27/327

(52) U.S. Cl. CPC G01N 27/327

(57) ABSTRACT

An electrode, comprising: a portion of MXene, the portion of MXene optionally being free of materials other than the MXene; and (a) a sealing material, the sealing material at least partially enclosing the portion of MXene, and the sealing material defining an opening through which a sensing region of the portion of MXene is exposed; or (b) a substrate, the portion of MXene being disposed on the substrate. An electrode, comprising: a fiber; a first coating superposed on the fiber, the first coating having a thickness and comprising a MXene composition, the MXene composition optionally comprising aligned MXene flakes, the first coating optionally having a uniform thickness along the length of the fiber; and a second coating superposed on the fiber.

### RELATED APPLICATIONS

The present application claims priority to and the benefit of U.S. patent application No. 63/584,555, “Transition Metal Carbides And/Or Nitrides (MXenes) For Electrochemical Sensing,” filed Sep. 22, 2023. All foregoing applications are incorporated herein by reference in their entireties for any and all purposes.

### TECHNICAL FIELD

The present application relates to the field of MXene materials and also to the field of electrochemical sensing.

### BACKGROUND

There is a long-felt need in the art for improved sensor device and related components.

### SUMMARY

In meeting the described long-felt needs in the art, the present disclosure provides an electrode, comprising: a portion of MXene, the portion of MXene optionally being free of materials other than the MXene; and (a) a sealing material, the sealing material at least partially enclosing the portion of MXene, and the sealing material defining an opening through which a sensing region of the portion of MXene is exposed; or (b) a substrate, the portion of MXene being disposed on the substrate.

Also provided is a method, comprising use of an electrode according to the present disclosure.

Further disclosed is a method, comprising contacting an electrode according to the present disclosure.

Additionally provided is a method, comprising fabricating an electrode according to the present disclosure.

Further provided is an electrode, comprising: a fiber; a first coating superposed on the fiber, the first coating having a thickness and comprising a MXene composition, the MXene composition optionally comprising aligned MXene flakes, the first coating optionally having a uniform thickness along the length of the fiber; and a second coating superposed on the fiber. It should be understood, however, that the second coating can be optional in some embodiments; in such embodiments, the electrode can comprise a fiber; a first coating superposed on the fiber, the first coating having a thickness and comprising a MXene composition, the MXene composition optionally comprising aligned MXene flakes, the first coating optionally having a uniform thickness along the length of the fiber, and the electrode being free of a second coating.

Also disclosed is a method, comprising contacting an electrode according to the present disclosure—for example, according to any one or more of Aspects 16-21—to a tissue.

Further provided is a method, comprising inserting an electrode according to the present disclosure—for example, according to any one of Aspects 16-21—into a subject.

Additionally provided is a method, comprising collecting a signal with an electrode according to the present disclosure—for example, according to any one of Aspects 16-21, the signal optionally relating to the evolution of a product.

Further provided is a method, comprising at least one of recording and stimulating physiologic activity with an electrode according to the present disclosure, for example according to any one of Aspects 16-21.

Also disclosed is a method, comprising removing a portion of an electrode according to the present disclosure—for example, any one of Aspects 16-21—so as to expose a cross-section of the fiber, the first coating, and the second coating of the electrode.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. Schematics of (a) MAX phase (Ti₃AlC₂) and (b) MXene (Ti₃C₂Tₓ) structures. c) X-ray diffraction patterns of Ti₃AlC₂ and Ti₃C₂Tₓ. d) Scanning electron microscopy images of basal-plane electrode (BPE) and its corresponding overlapping energy dispersive X-Ray spectroscopy (EDS) maps with (f) its respective spectra.

FIG. 2. Effect of Ti₃C₂Tₓ flake size. a) Size distribution of as-synthesized (L-flakes, d=1750 nm) and probe-sonicated (S-flakes, d=270 nm) MXene colloidal solutions. Inset: Scanning electron microscopy (SEM) images of large (top) and small (bottom) flakes colloidal solution. b) Cyclic voltammograms of Ti₃C₂Tₓ pristine electrodes fabricated with MXene colloids with large (L-) and small (S-) flakes in 5 mM ruthenium hexamine and 1 M KCl at a scan rate of 5 mV s⁻¹. c) Peak separation potential (□E, mV, mean±standard error, p<0.001, n=5) and (d) reduction peak current (I_{pc}, mA, mean±standard error, p<0.0001, n=5) of L- and S-flake electrodes.

FIG. 3. Effect of electrode thickness (m, a-b) or diameter (mm, c-d) of pristine Ti₃C₂Tₓ electrodes on electrochemical activity. Cyclic voltammograms (a, c) of 5 mM ruthenium hexamine in 1 M KCl at 5 mV s⁻¹. Corresponding ratios of faradaic (I_{f}) to capacitive (I_{c}) currents (b,d). (Mean±standard error, n=5)

FIG. 4. Effect of Ti₃C₂Tₓ flake orientation. a) Schematic of Ti₃C₂Tₓ structure highlighting its basal and edge planes. Scanning electron microscopy (SEM) micrographs of b) horizontal and c) cross-sectional plane of vacuum filtered films. The insets show optical images of their respective electrodes. d) Cyclic voltammograms of Ti₃C₂Tₓ basal-plane electrodes (BPE) and edge-plane electrodes (EPE) in 5 mM ruthenium hexamine and 1 M KCl at a scan rate of 5 mV s⁻¹. c) Reduction peak current (I_{pc}) and (f) peak separation potential (ΔE, mV) of BPEs and EPEs. (Mean±standard error, p<0.0001, n=6)

FIG. 5. a) Schematic of a film indicating electrode location and optical image of the corresponding electrodes. b) Representative cyclic voltammograms in 5 mM ruthenium hexamine and 1 M KCl at 20 mV s⁻¹ of electrodes in different locations. c) Cathodic peak current (I_{pc}, uA) and d) peak separation potential (□E, mV). (Mean±standard error, p<0.05, n=5)

FIG. 6. a) Schematic showing the difference between the control electrode, (Configuration A) the bent electrode during measurement and (Configuration B) an outward bend out of 100 bends (50 inwards and 50 outwards) along the electrode diameter before measurement. b) Cyclic voltammograms of electrodes in 5 mM ruthenium hexamine in 1 M potassium chloride at 20 mV s⁻¹. c) Reduction peak current (I_{pc}, mA) and (d) redox peaks separation (mV) of cyclic voltammograms. (Mean±standard error, n=6)

FIG. 7. a) Spray-coated Ti₃C₂Tₓ electrodes on top of paper-printed logos. b) Transmittance (%) spectra in the UV-visible region. c) Cyclic voltammogram (CVs) of 5 mM ruthenium hexamine in 1 M potassium chloride at 20 mV s⁻¹. d) Reduction peak current (mA cm⁻²) and peak separation potential (V) of corresponding CVs (Mean±standard error, p<0.0001, n=4).

FIG. 8. Batch reproducibility. a) Cyclic voltammograms of representative Ti₃C₂Tₓ electrodes from 5 fabricated batches. CVs ran in 5 mM ruthenium hexamine in 1 M potassium chloride at 20 mV s⁻¹. b) Reduction peak current (I_{pc}, mA) and (c) redox peaks separation (DE, mV) of cyclic voltammograms.

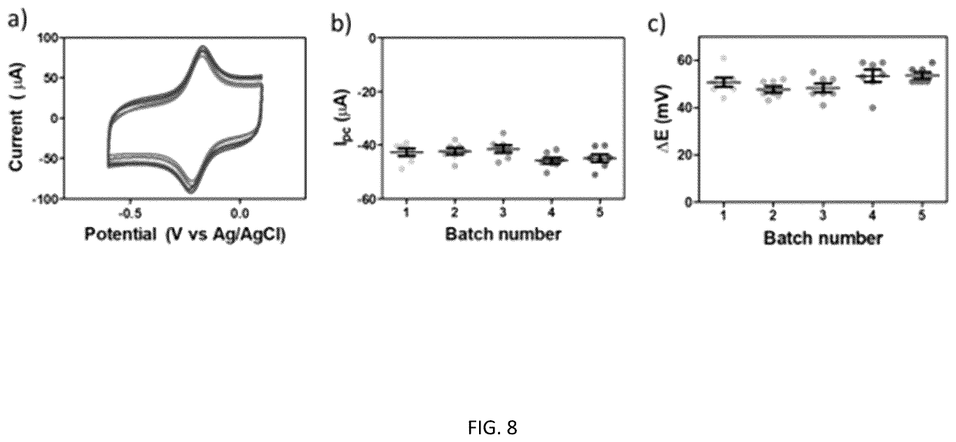

FIG. 9. Electrode fabrication. (1) Vacuum filtration of MXene colloidal solution. (2) Self-standing film (3) cut and (4-7) Kapton encapsulation.

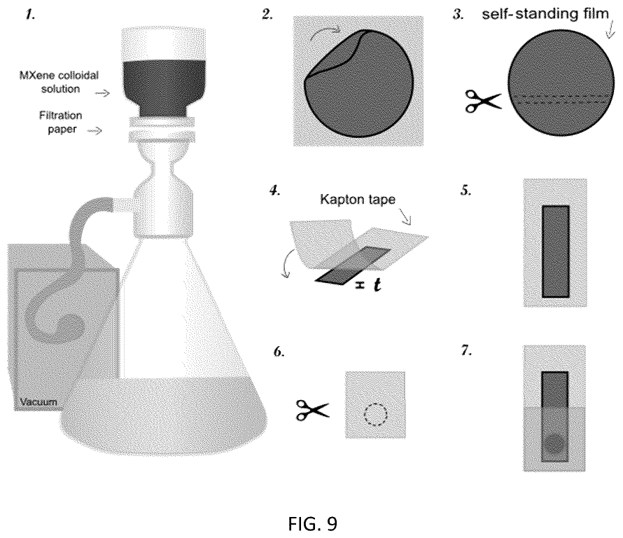

FIG. 10. Schematics of the fiber electrode fabrication and example applications. (a) Schematic of the dip coating process consisting of a nylon filament running through a dispersion of single-layer MXene flakes. During this process, MXene flakes are aligned in the axial direction by the shear force present in the meniscus. (b) Schematics and digital photographs of the nylon filaments, MXene-coated nylon filaments, and Parylene-coated electrodes. (c) Schematic of cut electrodes with the cross-section of the MXene coating exposed and their demonstrated capabilities in this work. (d) Example, non-limiting applications of the MXene electrodes in various biological systems, including cells, organoids, brains, and muscles.

FIG. 11. Tunability of the facile filament dip coating process. (a) Parametric study of linear resistance with the filament diameter, MXene concentration, and drawing speed. Cross-sectional SEM images and EDS maps of 300 μm nylon filament coated using (b) 110 and (c) 10 mg/mL MXene solution, as well as (d) 100 μm filament coated using 110 mg/mL MXene solution. Samples were coated at 15 mm/s drawing speed. EDS images are in the net count with brightness increases of 60, 90, and 60%, respectively.

FIG. 12. Electrode characterization in common electrolytes and analytes for biological applications. Cyclic voltammograms (CVs) of 300 μm electrodes as a function of MXene concentration (10 and 110 mg/mL) in 5 mM RuHex in 1 M KCl at 20 mV/s and reproducibility between (a) different electrodes and (b) multiple cuts of the same electrode. (c) CVs at 100 mV/s and (d) impedance modulus of 110 mg/mL MXene coated electrodes as a function of frequency for 3 different diameters (300, 100, and 28 μm) in 1×PBS. Data are plotted as average values with shaded regions corresponding to SDs. (e) CVs before and after knotting of 100 μm electrodes at 100 mV/s in PBS. (f) Average impedance and SD at 1 KHz in 1×PBS over 1000 insertions (100 μm electrodes coated with 110 mg/mL inserted to a depth of 3-4 cm) into 0.6 wt % agarose. The drawing speed for all tested electrodes is 15 cm/s.

FIG. 13. H₂O₂ sensing and ex vivo demonstration in bladder urothelium. (a) Amperometry at −650 mV (vs Ag/AgCl) with additions of H₂O₂ (20, 50, 100, 200, and 500 μM) every 50 s. The electrodes are 300 μm electrodes coated with 10 mg/mL MXene and 110 mg/mL MXene, as well as carbon fiber electrodes. (b) Calibration curve of H₂O₂. (c) Stability of response signal during 30 min-1 mM of H₂O₂ at t=50 s using amperometry. (d) Stability of response to 1 mM H₂O₂ over days. (a-d) Share the same legend. (e) Optical microscopy image of the bladder urothelium, where a 10 mg/mL MXene fiber microelectrode was placed in contact of the urothelium, which was constantly perfused with H-medium. Peroxide production was stimulated with 200 nM thapsigargin using microperfusion. (f) Amperometric detection of peroxide using MXene fiber microelectrode at −650 mV vs Ag/AgCl. The arrow indicates the point at which either H-medium (black trace) or 200 nM thapsigargin (brown trace) was microperfused onto the urothelium.

FIG. 14. MXene-nylon microfiber electrodes can record and stimulate electrophysiological activity. (a) The cathodic potential excursion E_{c} values plotted against injected cathodic current amplitude for 110 mg/mL MXene, 15 mm/s coated electrodes with diameters of 300, 100, and 28 μm. The cathodic voltage limit of MXene electrodes is displayed as a dashed line and SD as shadows (n=3). (b) The same plot on a scaled X-axis for 28 μm diameter electrodes. (c) The maximum charge injected (C) across electrodes of 3 different sizes. (d) Schematic of the in vivo stimulation and recording experiment with a pair of MXene electrodes placed on the sciatic nerve as the bipolar stimulation electrodes and another MXene electrode on the surface of the tibialis anterior muscle as the recording electrode. (c) Representative evoked electromyography (EMG) potential at the tibialis anterior as recorded by a 300 μm MXene electrode when the sciatic nerve is stimulated with a pair of 300 μm MXene electrodes at a cathodic current of 200 μA. Gray plots denote the individual pulses (n=10), and the purple plots denote the average of individual traces. (f) Peak-to-peak evoked EMG as a function of stimulating current amplitude measured using 100 and 300 μm MXene microelectrodes.

FIG. 15. Surface electromyography (sEMG) recordings using MXene-coated nylon fiber. (a) Schematic illustrating the recording of sEMG activity during contraction of the biceps using a representative MXene-coated nylon fiber. (b) sEMG activity and (c) root-mean-square of the sEMG activity recorded at the biceps using a 300 μm diameter MXene-coated nylon fiber electrode. The marked black lines denote muscle contractions.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments of aspects can be combined with any part or parts of any one or more other embodiments or aspects.

Since the discovery of two-dimensional (2D) transition metal carbides, nitrides and carbonitrides (MXenes), electrochemical studies have mostly focused on the development of energy storage devices. Since 2017, enhanced sensitivity and decreased limit of detection for multiple analytes have been reported by the incorporation of MXenes into electrochemical sensors. However, the precise MXene contribution to electrochemical sensing is unknown due to a lack of studies using pristine MXene electrodes. Herein, we developed pristine Ti₃C₂Tₓ MXene electrochemical sensors, where we explored the impact of material characteristics (flake size, flake orientation, film geometry, and uniformity of MXene film) on the electrochemical activity of the outer sphere redox probe ruthenium hexamine using cyclic voltammetry. We have demonstrated that large flakes were beneficial for higher current response and faster electron transfer kinetics of the electrode compared to small flakes. The reduction in electrode thickness from 8 mm to 2 mm increased the faradaic-to-capacitance ratio. Inter- and intra-batch reproducibility was demonstrated with no significant differences between electrode performances. Pristine Ti₃C₂Tₓ electrodes, without the need for current collectors or other electroactive species, can act as flexible and transparent sensors which are key elements for the translation of MXene electrodes to specialized real-life sensing applications.

Since their discovery in 2011, MXenes have been explored in a wide range of applications due to their tailorable properties.^{1,2} To date, most MXene electrochemical studies have focused on energy storage uses because of their excellent capability of ion intercalation and pseudocapacitance properties.³ However, the rare combination of electroactive surface area with high electrical conductivity, reaching over 20,000 S cm⁻¹ in multilayer films made of aligned MXene flakes, makes Ti₃C₂Tₓ also promising for analytical electrochemistry.⁴ Most reported electrochemical MXene-based sensors are chemically-modified electrodes in which MXene is used in combination with various other components, such as gold or silver nanoparticles, or other nanomaterials added to the surface of a glassy carbon (GC) electrode.⁵⁻⁷ These studies have shown that incorporating MXene into the working electrode increases sensitivity to different analytes such as redox probes, neurotransmitters, and environmental pollutants.^{8.9} However, the design of these electrochemical sensors makes it difficult to determine the contribution of MXene to the faradaic response resulting from the reduction or oxidation of the analyte at the electrode surface.

The large family of MXenes currently consists of more than 30 stoichiometric compositions and dozens of solid solutions, providing a diverse range of surface chemistries and structures.³ Following synthesis, different processing steps and fabrication protocols can tailor other characteristics of these two-dimensional (2D) flakes including flake size and orientation. In various fields of study, extensive research has been conducted to investigate the influence of synthesis, processing and fabrication methods, on the performance of MXene-based devices. For instance, large flakes generally result in high conductivity, whereas small flakes allow for better electrolyte accessibility for energy storage devices.¹⁰ Furthermore, for electrocatalysis, differential adsorption behavior has been observed between the edge and basal plane of MXenes.¹¹ Molybdenum carbide (Mo₂CTₓ) has been shown to have higher catalytic activity at the edges similar to other 2D nanomaterials, but its basal planes still maintained the activity, unlike dichalcogenides.^{12.13} Directional ion transport has been demonstrated for aligned MXene flakes, highlighting the importance of flake orientation.¹⁴ While material characteristics have been investigated across a range of applications, the understanding of their impact on the electrochemical behavior of sensors remains limited. The removal of other electroactive materials, by developing pristine MXene electrodes, should enable the optimization of both the material and electrode parameters for electrochemical sensors.

Utilizing pristine Ti₃C₂Tₓ also offers key advantages for electrochemical sensors. For example, the low absorption in visible range wavelengths by MXene flakes can provide the ability to make transparent thin film electrodes. Transparent Ti₃C₂Tₓ coatings have already been used for accommodative intraocular lenses, energy storage devices and antennas.^{15.16} The mechanical properties of Ti₃C₂Tₓ flakes are also advantageous when compared to other sensing materials. The tensile stress and Young's modulus of pristine Ti₃C₂Tₓ films have been reported to reach up to 568 MPa and 20.6 GPa, respectively, with a breaking elongation of 3.2%.¹⁷ Notably, the tensile stress of pristine Ti₃C₂Tₓ films surpasses that reported for reduced graphene oxide films (478 MPa).¹⁸ The flexibility of MXene films provides the ability to interface with various surfaces as electrochemical sensors to be used for MXene-based wearables.¹⁹ Given the unique material properties of MXene, the aim of this study was to explore the use of pristine Ti₃C₂Tₓ MXene for electrochemical sensing. Voltammetric studies were conducted using a standard redox probe to explore the electrochemical performance of Ti₃C₂Tₓ MXene by varying flake size and orientation (basal vs edge plane of freestanding film electrode), and electrode geometries (thickness and geometrical surface area). The electrochemical activity of transparent and flexible Ti₃C₂Tₓ electrodes was also explored to highlight the scope of aligned MXene films for electrochemical sensing. It should be understood that Ti₃C₂Tₓ is an illustrative MXene, as the disclosed technology is not limited to Ti₃C₂Tₓ or any other particular MXene.

### Results and Discussion

### MXene Synthesis and Characterization

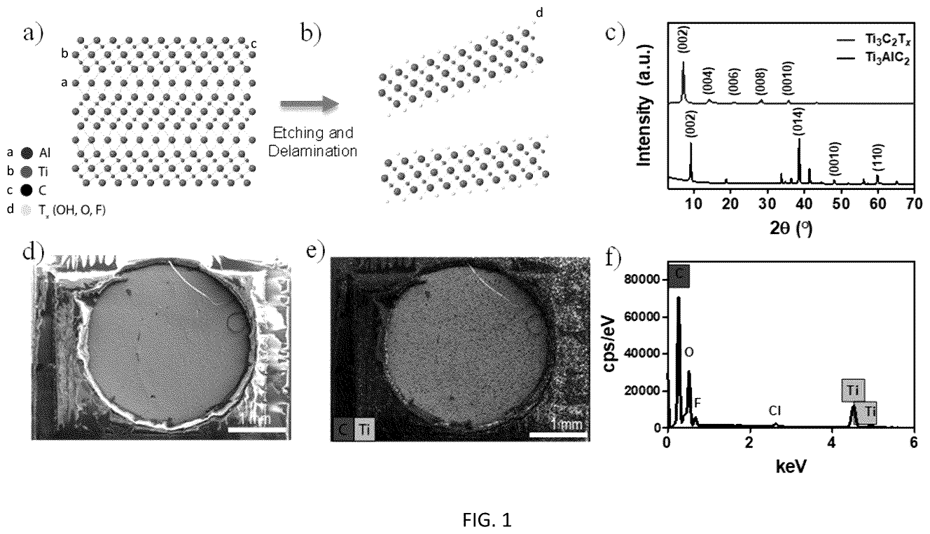

Delaminated Ti₃C₂Tₓ flakes were synthesized from its precursor material, Ti₃AlC₂ MAX phase, via selective etching of Al layers following a previously published protocol (FIG. 1a,b).⁴ The successful synthesis was confirmed using X-ray diffraction (XRD, FIG. 1c) of a Ti₃C₂Tₓ freestanding film produced via vacuum-assisted filtration. The shift of the (002) peak and the corresponding increase in d-spacing from 9.5 Å for Ti₃AlC₂ to 12.3 Å for the freestanding Ti₃C₂Tₓ film is attributed to the termination groups (Tₓ) of delaminated Ti₃C₂Tₓ and intercalated water molecules. In addition, only (001) peaks are present for the film sample, indicating that the Ti₃C₂Tₓ sheets are all aligned along the basal direction with no residual Ti₃AlC₂ or other impurities present.²⁰ This confirms the removal of the Al layer, delamination of single MXene flakes with lithium ions, and suitability of the produced film to study the Ti₃C₂Tₓ flake orientation.

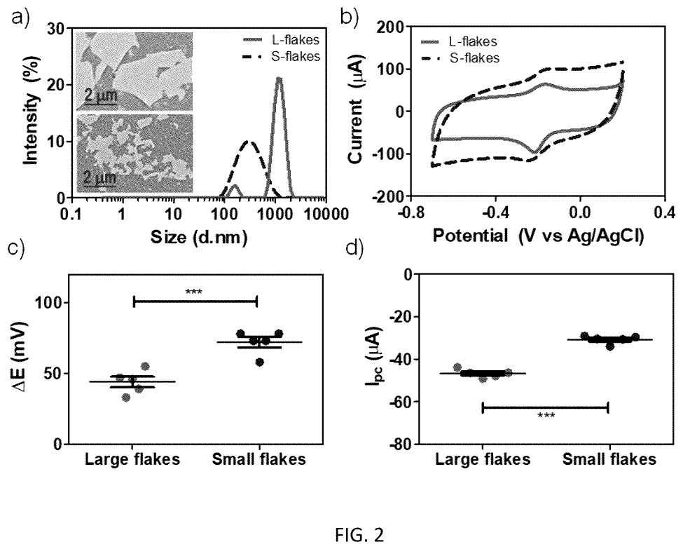

FIG. 1d shows the scanning electron microscopy (SEM) image of an electrode for electrochemical sensing prepared from the freestanding Ti₃C₂ Tₓ film by encapsulating it in a Kapton tape with a hole of a predetermined diameter. Electrode fabrication is described in the Experimental section. The Ti₃C₂Tₓ basal plane was exposed to the analyte, thus, it was referred to as a basal-plane electrode (BPE). Energy dispersive X-ray spectroscopy (EDS) mapping (FIG. 1c) showed the presence of Ti and C from the core Ti₃C₂Tₓ structure. Moreover, chlorine, fluorine and oxygen were identified (FIG. 2f) and attributed to the termination groups acquired during wet synthesis. These findings indicated the typical for Ti₃C₂Tₓ composition of the BPE electrodes.

### Impact of MXene Flake Size on Electrode Electroactivity

To investigate the impact of flake size on the electrochemical activity of pristine MXene, BPEs containing different flake sizes were fabricated. The starting flake size distributions were measured via dynamic light scattering (DLS) and are shown in FIG. 2a, where the as-synthesized Ti₃C₂Tₓ colloidal solution is referred to as “large flakes” (L-flakes) with an average hydrodynamic diameter of 1750 nm±65 nm and a polydispersity index (PDI) of 0.542. The flake size is typically inherited from the precursor Ti₃AlC₂ phase crystal size and the high polydispersity attributed to the synthesis conditions whereby flakes are broken into smaller size.²¹ Probe sonication reduced the flake (S-flakes) as shown in their DLS distribution (FIG. 2a) to an average size of 270 nm±2 nm with a PDI of 0.219. The inset of FIG. 2a shows SEM images of the flakes deposited on porous alumina substrate, clearly illustrating the difference in size between L- and S-flakes.

L- and S-flake electrodes were investigated using the outer sphere redox probe, ruthenium hexamine using cyclic voltammetry (FIG. 2b). The combination of a capacitive window with the faradaic peaks observed for ruthenium hexamine shows the pseudocapacitive behavior of pristine Ti₃C₂Tₓ electrodes.³ The S-flake electrode cyclic voltammogram (CV) shows a wider capacitive box which is likely due to greater ion diffusion as previously reported for small Ti₃C₂Tₓ flakes.¹⁰ A slanted voltammogram for the S-flake electrodes was observed. This, combined with the significant increase in the difference between the cathodic and anodic peak potentials (ΔE) in S-flake electrodes when compared to L-flake electrodes (p<0.0001, n=5, FIG. 2c), suggests higher resistivity for S-flake electrodes. The small flakes within the S-flake electrodes likely cause an increase in flake-to-flake contact resistance as previously reported.¹⁰ The cathodic current peak for L-flake electrodes was significantly higher than that and S-flake electrodes (p<0.001, n=5, FIG. 2d). The smaller peak current observed for the S-flake electrode is likely due to the slanted voltammogram obscuring the faradaic current and making its determination less accurate. In summary, L-flake electrodes showing a higher peak current are more sensitive due to using outer-sphere redox probes in addition to their better kinetics.

### Impact of Geometrical Size and Thickness of the Electrode

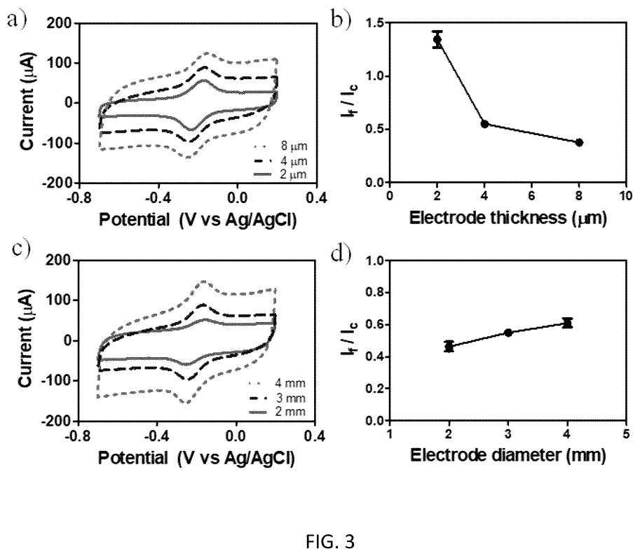

FIG. 3a shows CVs of BPEs with varying thicknesses (2, 4, and 8 mm) developed with the L-flake colloidal solution. There was a significant increase in ΔE (63±3 mV for 2 μm, 76±8 mV for 4 μm, and 88±6 mV for 8 μm; p<0.05, n=6) with increased electrode thickness. This suggests an increase in resistivity and slower kinetics of the MXene film with increased thickness. Moreover, from the CV of ruthenium hexamine, the capacitive box increased with increasing thickness, but no noticeable difference was observed in the faradaic current (FIG. 3a). The faradaic current was then divided by the capacitive current to understand the impact of electrode thickness on the Faradaic signal. FIG. 3b shows that with increased electrode thickness, the ratio of faradaic-to-capacitive current decreased. This is most likely due to the increased thickness of the electrode providing more pathways for positively charged potassium ions from the electrolyte to penetrate the electrode structure and occupy electroactive surface sites of MXene flakes within the electrode, thus enhancing the capacitive current, whilst the redox probe only accesses the electrode surface.

FIG. 3c shows voltammograms of electrodes made with varying diameters (2, 3 and 4 mm) with constant thickness (4 m). There was no significant difference in ΔE (59.2±4 mV for 2 mm, 56.2±8 mV for 3 mm and 63±4 mV for 4 mm) with increased electrode diameter when performing a one-way ANOVA analysis. Furthermore, FIG. 3d shows that the ratio of faradaic-to-capacitive current increases with an increase in surface area. This most likely is due to increased surface roughness when increasing the geometrical surface area of the electrode. MXene electrochemical sensors of any diameter and with minimal thickness would provide the greatest faradaic-to-capacitive current ratio.

### Impact of Flake Orientation

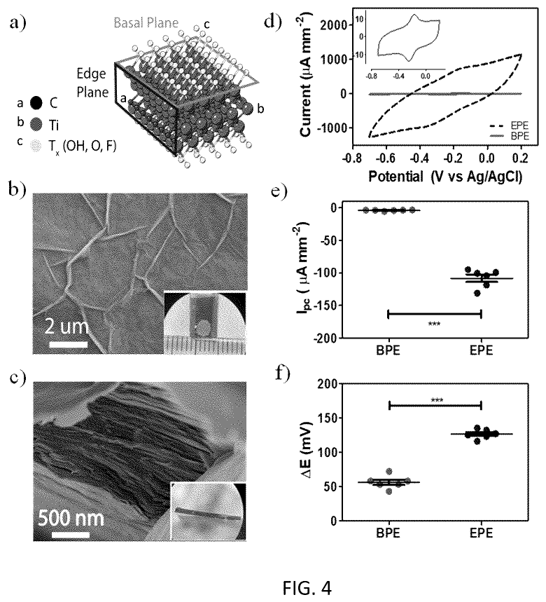

Due to the 2D structure of MXenes, the sites exposed at the electrode surface will vary by chemical structure. The outer layers of Ti₃C₂Tₓ MXene comprise titanium atoms terminated by oxide, hydroxide, and fluoride groups on the basal plane, while little is known about the termination groups at the edge plane in which carbide layers are found between the titanium layers (FIG. 4a). To investigate the differences in electroactivity of flake orientation, two electrode designs were prepared taking advantage of the organized structure (XRD, FIG. 1c) of vacuum-filtered Ti₃C₂Tₓ films. The SEM image of the top section of the film (FIG. 4b) shows wrinkles of the overlapping layers while the cross-sectional SEM image of the film (FIG. 4c) shows the stack of layers exposing the edges of the Ti₃C₂Tₓ flakes. The inset of FIGS. 4b and c present optical images of the electrodes fabricated, predominantly exposing the basal plane of Ti₃C₂ Tₓ flakes (basal-plane electrode, BPE) or the edge-plane of Ti₃C₂Tₓ flakes (edge-plane electrode, EPE).

CVs of BPEs and EPEs were obtained in ruthenium hexamine (FIG. 4d) and the current response was normalized by the electrode geometrical area. The normalized cathodic peak current of the EPE was significantly higher than that of the BPE (p<0.0001, n=6, FIG. 4c). This is most likely due to two factors. First, the EPE being an ultramicroband electrode, with thickness (or width) below 25 □m, resulted in a two-dimensional diffusion system different from that of BPE, which is a macroelectrode.²² Second, the nonuniformity of the edges exposing variations in surface area between the Ti₃C₂Tₓ flakes, as seen in the SEM image of FIG. 4c, likely leads to a poor representation of the electroactive surface area, resulting in a larger experimental current observed for EPE. The ΔE of BPEs were significantly smaller (p<0.0001, n=6) than that of EPE (FIG. 4f). While BPEs showed better kinetics than the EPEs, the high current density of EPEs highlight their utility in sensors with high sensitivity.

### Uniformity of Film and Electrode Reproducibility

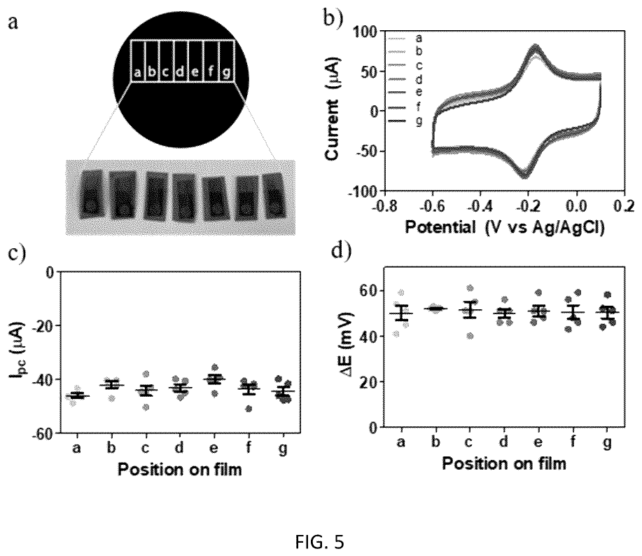

To test the reproducibility of the electrodes, we studied the uniformity of the MXene film from which electrodes were made. The schematic in FIG. 5a shows the location from which the electrodes (optical photograph) were obtained. The representative CVs of these electrodes in ruthenium hexamine are shown in FIG. 5b. No significant difference (p<0.05, n−6) was observed for the cathodic peak current (FIG. 5c) and ΔE (FIG. 5d). This suggests that a uniform film was developed when using vacuum-assisted filtration. It has been shown that blade coating allows the manufacturing of large-area films with a well-defined thickness, which is required for sensing studies, as highlighted in the previous section.²³

The variation between different batches of these MXene electrodes was also investigated, and no significant difference in cathodic peak current and ΔE was observed between different batches of MXene electrodes. These findings highlight the MXene electrochemical sensors can be made with high reproducibility.

Flexible Ti₃C₂Tₓ Electrodes

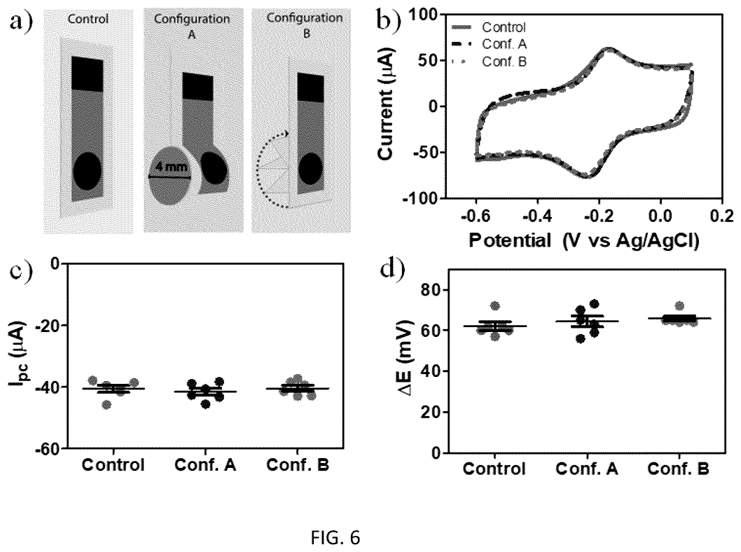

To test the use of pristine Ti₃C₂Tₓ electrodes as flexible sensors, two different investigations were conducted against a control (FIG. 6a, Control). In the first condition (FIG. 6a, Condition A), the BPE MXene electrode was bent around a 4 mm cylinder and measurements of ruthenium hexamine were conducted. In the second condition (FIG. 6a, Condition B), the response to ruthenium hexamine was tested after the electrode was bent 100 times (50 times outwards and 50 times inwards) along the electrode diameter. FIG. 6b shows representative CVs of ruthenium hexamine under the two conditions and control electrode, where no particular differences were observed. There was no significant difference in the cathodic peak current (FIG. 6c) and ΔE (FIG. 6d) when the electrode was measured during flexible conditions when compared to control BPE electrodes. These findings are consistent with previous work in which micro-supercapacitors utilizing pristine MXene demonstrated remarkable flexibility, retaining 85% of their capacitance even after undergoing 1600 bends.²⁴ These results highlight the ability of pristine MXene electrochemical sensors to provide reproducible responses even when flexed, making them well-suited for sensing applications requiring functional stability under motion.^{25,26}

Transparent Ti₃C₂Tₓ Electrode

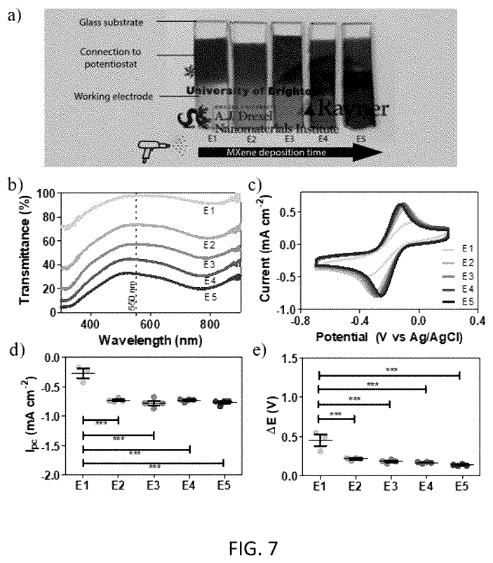

To explore the use of Ti₃C₂Tₓ as transparent electrochemical sensors, thin-film electrodes were developed by spray-coating MXene onto glass substrates. Increasing deposition times resulted in decreasing optical transparency (FIG. 7a). The thickness of the top section of the electrodes was deliberately increased to ensure a good electrical connection. The transmittance spectra of the resulting electrodes indicate an absorption peak at around 780 nm (FIG. 7b), which is consistent with the plasmonic absorption peak characteristic of Ti₃C₂Tₓ.²⁷ The transmittance is also reduced at lower wavelengths due to inter-band transitions.²⁸ Nonetheless, the visible transparency of each electrode was evaluated at the standard reference point of 550 nm, resulting in decreasing values of 97.9% (a), 73.8% (b), 57.4% (c), 44.3% (d), and 32.6% (c) with increasing MXene deposition time. Based on the previously reported relationship between the absorbance and thickness of Ti₃C₂Tₓ coatings, the spin-coated MXene electrodes were estimated to have thicknesses of 1.8 nm, 26.4 nm, 48.2 nm, 70.8 nm, and 97.6 nm, respectively, with increasing deposition time.²⁹

FIG. 7c shows the CVs of ruthenium hexamine. For comparison between the impact of degrees of transparency of the electrodes in electrochemical behavior, the current was normalized by the electrode surface area that was in contact with the electrolyte. The redox peaks observed at around-0.2 V confirmed that Ti₃C₂Tₓ can be used as a transparent electrode for the electrochemical reduction and oxidation of electroactive species, suggesting their use for transparent sensors. These CVs also demonstrate the ideal faradaic-to-capacitive current ratio previously discussed, in which faradaic current dominates the response. FIG. 7d shows that the reduction peak current (mA cm⁻²) of the transparent electrodes remained stable (p<0.0001, n=4) at a transmittance of 73.8% and below (electrodes E2-5). These findings suggest that comparable sensitivities can be attained at a relatively high transmittance of 73.8%. The decrease in electrochemical performance of the thinnest electrode studied (E1) is probably due to a non-uniform coverage considering the estimated thickness of 1.8 nm is close to monolayer coverage. A similar conclusion is obtained from FIG. 7e in which the separation peak potential (V) is not significantly decreased beyond an electrode transmittance of 73.8%. There is a slight trend of decreasing □E with increasing thickness which is attributed to an increase in electron pathways within the coating. This work demonstrates the use of Ti₃C₂Tₓ in transparent sensors without compromising electrode performance.

### Conclusion

This work showcases the performance of a pristine Ti₃C₂Tₓ electrochemical sensor. MXene films were able to make highly reproducible batches of electrodes. Larger flakes and thin film electrochemical sensors provided the greatest faradaic signal on top of the capacitive current. MXenes films were used to make transparent and flexible electrochemical sensors that had excellent electrochemical activity. Overall, our findings highlight key considerations on how to make MXene electrochemical sensors that can provide enhanced performance. MXene electrochemical sensors have wide-ranging benefits in wearable, portable and implantable sensing devices due to the unique properties of these 2D materials.

### Experimental Section

### Materials

Hydrofluoric acid (Acros Organics, 50 wt %), concentrated hydrochloric acid (Fisher Scientific, 37 wt %), titanium carbide (Alfa Aesar, 99.5%, 2 μm), titanium (Alfa Aesar, 99.5%, 325 mesh), aluminum (Alfa Aesar, 99.5%, 325 mesh), lithium chloride (LiCl), potassium chloride (KCl) and hexaammineruthenium (III) chloride ((Ru(NH₃)₆Cl₃, Sigma-Aldrich) were used as received. Polyimide electrical tape was purchased from RS Components, Ltd. (UK).

Synthesis of Ti₃C₂ Tₓ MXene

Ti₃C₂Tₓ was synthesized through the selective etching of aluminum from the stoichiometric MAX phase, Ti₃AlC₂, following a previously published protocol.⁴ In short, Ti₃AlC₂ was synthesized from TiC, Ti, and Al powders with excess of metals (2:1:1 mass ratio of TiC, Ti, and Al) via annealing at 1380° C. for 2 hours under Ar atmosphere. The resulting mixture was powdered, washed with 9 M hydrochloric acid (HCl) to remove intermetallic impurities, dried and sieved to obtain particles below 38 μm. The Ti₃AlC₂ MAX phase was etched using hydrofluoric acid and hydrochloric acid (2 mL HF, 12 mL HCl, 6 mL H₂O per 1 g of MAX) over 24 hours at 35° C.³⁰ The multi-layered Ti₃C₂Tₓ was washed through centrifugation (5 min, 3500 rpm) using deionized (DI) water until neutral pH. Next, the multi-layered Ti₃C₂Tₓ was stirred at room temperature for 24 hours with 1 g LiCl for delamination. The MXene/LiCl mixture was centrifuged for 5 min at 3500 rpm and the supernatant was discarded. MXene was redispersed in water by shaking and centrifuged for 15 min at 3500 rpm, after which the supernatant containing predominantly single-layer MXene flakes was collected. The redispersion-shaking-centrifugation steps were continued until the supernatant was transparent, indicating a low concentration of MXene. Finally, the delaminated Ti₃C₂Tₓ was concentrated by centrifugation at 10,000 rpm for 10 min and stored in Ar vials in a refrigerator until used.

### Flake Size Reduction and Characterization

To reduce the size of Ti₃C₂Tₓ flakes, probe sonication (50% amplitude, 8:2 sec ON: OFF) was performed over 20 mins using an ice bath to avoid heating the MXene colloidal solutions. To estimate flake size dimensions and determine the polydispersity index, Dynamic Light Scattering (DLS) was used with a 90-degree scattering optics spectrometer was utilized (Zetasizer Nano ZS, Malvern Panalytical, UK). The sample of interest was diluted until visually translucent and 1 mL of a MXene sample was pipetted into a polystyrene cuvette. Three measurements were averaged for each sample. To confirm the flake size, scanning electron microscopy (Zeiss Supra 50VP scanning electron microscope) images were recorded of single flakes drop-casted on a porous anodic alumina membrane.

### Electrode Fabrication

A known volume of as-synthesized Ti₃C₂Tₓ colloidal solution (0.7 mg/mL) were vacuum filtered using a hydrophilic polyvinylidiene difluoride (PVDF) membrane (0.22 mm, Merk Chemicals LTD). The MXene films were left to air dry until they could be removed from the filtration paper and were stored until further used (SI 2). The film thicknesses were measured using a digimatic micrometer (absolute d2, Multitoyo). Kapton tape was utilized to encapsulate the film and maintain a constant electrode area (SI 2). Two electrode designs were developed. To consider the horizontal orientation of the films, a piece of Kapton tape had been previously hole-punched with a determined diameter (d) to expose the top area of the film. This electrode design was named basal-plane electrode (BPE). To consider the vertical alignment of flakes, once the MXene section of determined length (1) was encapsulated by the two pieces of Kapton tape, a cross-sectional area of MXene was exposed by performing a cut parallel to the width of the MXene. This electrode design was named edge-plane electrode (EPE).

To fabricate transparent electrodes, thin Ti₃C₂Tₓ films were deposited onto a glass substrate. As-synthesized MXene colloidal solution (5 mg mL⁻¹) was spray-coated over 40 mins. In order to achieve electrodes with varying thicknesses, the lower section of the substrate (working electrode) was masked at specific time intervals (5, 10, 15, 20, and 25 minutes) during the deposition process. Meanwhile, the deposition of Ti₃C₂Tₓ over the higher section of the substrates continued uninterrupted throughout the entire deposition period.

### X-Ray Diffraction Measurements

X-ray diffraction (XRD, Rigaku Smartlab) was recorded in pressed Ti₃AlC₂ MAX powder and vacuum-filtered Ti₃C₂Tₓ film. For both, the Smartlab was operated at 40 kV and 30 mA with Cu Kα radiation. For the MAX powder, the scan was performed from 3-90°, with a step scan of 0.04°, and a holding time of 0.5 s/step. While, for the MXene film, the scan was performed from 3-70°, with a step scan of 0.01°, and a holding time of 0.75 s/step.

### Scanning Electron Microscopy Measurements

Sample preparation for scanning electron microscopy (SEM) was performed by cutting a vacuum-filtered film and placing it either horizontally or vertically on the bolt sample holder to obtain a top or cross-sectional image, respectively. SEM images were obtained with a Zeiss Sigma field emission gun (FEG) SEM. These secondary electron images were generated using an Everhart-Thornley secondary electron detector in the FEG-SEM. Chemical compositions of the MXene electrodes were determined using an Aztec Energy Dispersive X-ray Spectroscopy (EDS) system (Oxford Instruments), equipped with an X-Max 80 X-ray detector. For SEM imaging and EDS mapping, the SEM/EDS was operated at 10 kV accelerating voltage in a high vacuum.

### UV-Vis Spectroscopy Measurements

To characterize the transparency of spray-coated electrodes, their UV-vis spectra was recorded using the Evolution 201 UV-vis spectrophotometer (Thermo Scientific, USA) scanning from 200-1000 nm. An uncoated glass substrate was utilized as the blank sample.

### Electrochemical Measurements

Electrochemical studies were performed using a CH 760E potentiostat (CH instruments, Texas). The three-electrode system used consisted of an Ag|AgCl (3 M KCl) reference electrode, a platinum wire auxiliary electrode and pristine Ti₃C₂Tₓ as the working electrode. Electrodes were characterized using 5 mM ruthenium hexamine in 1 M KCl using cyclic voltammetry. CVs were recorded in potential windows between +200 and −700 mV at a scan rate of 5 or 20 mV s⁻¹. Measurements of the capacitance were measured in 1 M KCl with the same electrochemical parameters.

### Data Analysis

The cyclic voltammetry measurements were analyzed for anodic/cathodic peak potential, the difference between the anodic and cathodic peak potential (ΔE) and current were obtained from the voltammograms using CHI 760E software. The geometric surface area (A, cm²) of the film electrodes was calculated using the diameter or the length and thickness of the electrodes. The geometric surface area of spray-coated electrodes was determined using Image J by selecting the wet area of the electrode that was in contact with the electrolyte. All statistical analyses were performed using GraphPad Prism 7, a statistical software. The data underwent analysis using either a one-way ANOVA with Tukey's multiple comparison test or an unpaired t-test, with a significance level set at p<0.05. The standard error of the mean was used to represent the error.

### REFERENCES

(1) Naguib, M.; Kurtoglu, M.; Presser, V.; Lu, J.; Niu, J.; Heon, M.; Hultman, L.; Gogotsi, Y.; Barsoum, M. W. Two-Dimensional Nanocrystals Produced by Exfoliation of Ti₃AlC₂. Adv. Mater. 2011, 23 (37), 4248-4253.
(2) Mohammadi, A. V.; Rosen, J.; Gogotsi, Y. The World of Two-Dimensional Carbides and Nitrides (MXenes). Science. 2021, p 6547.
(3) Li, X.; Huang, Z.; Shuck, C. E.; Liang, G.; Gogotsi, Y.; Zhi, C. MXene Chemistry, Electrochemistry and Energy Storage Applications. Nat. Rev. Chem. 2022, 6, 389-404.
(4) Mathis, T. S.; Maleski, K.; Goad, A.; Sarycheva, A.; Anayee, M.; Foucher, A. C.; Hantanasirisakul, K.; Shuck, C. E.; Stach, E. A.; Gogotsi, Y. Modified MAX Phase Synthesis for Environmentally Stable and Highly Conductive Ti₃C₂ MXene. ACS Nano 2021, 15 (4), 6420-6429.
(5) Rakhi, R. B.; Nayuk, P.; Xia, C.; Alshareef, H. N. Novel Amperometric Glucose Biosensor Based on MXene Nanocomposite. Sci. Rep. 2016, 6, 36422.
(6) Lorencova, L.; Bertok, T.; Filip, J.; Jerigova, M.; Velic, D.; Kasak, P.; Mahmoud, K. A.; Tkac, J. Highly Stable Ti₃C₂Tₓ (MXene)/Pt Nanoparticles-Modified Glassy Carbon Electrode for H₂O₂ and Small Molecules Sensing Applications. Sensors Actuators, B Chem. 2018, 263, 360-368.
(7) Kalambate, P. K.; Gadhari, N. S.; Li, X.; Rao, Z.; Navale, S. T.; Shen, Y.; Patil, V. R.; Huang, Y. Recent Advances in MXene-Based Electrochemical Sensors and Biosensors. Trends Anal. Chem. 2019, 120, 115643.
(8) Liu, H.; Duan, C.; Yang, C.; Shen, W.; Wang, F.; Zhu, Z. A Novel Nitrite Biosensor Based on the Direct Electrochemistry of Hemoglobin Immobilized on MXene-Ti₃C₂. Sensors Actuators, B Chem. 2015, 218 (5), 1343.
(9) Amara, U.; Sarfraz, B.; Mahmood, K.; Mehran, M. T.; Muhammad, N.; Hayat, A.; Nawaz, M. H. Fabrication of Ionic Liquid Stabilized MXene Interface for Electrochemical Dopamine Detection. Microchim. Acta 2022, 189 (2), 64.
(10) Maleski, K.; Ren, C. E.; Zhao, M. Q.; Anasori, B.; Gogotsi, Y. Size-Dependent Physical and Electrochemical Properties of Two-Dimensional MXene Flakes. ACS Appl. Mater. Interfaces 2018, 10 (29), 24491-24498.
(11) Luo, Y.; Chen, G. F.; Ding, L.; Chen, X.; Ding, L. X.; Wang, H. Efficient Electrocatalytic N₂ Fixation with MXene under Ambient Conditions. Joule 2019, 3 (1), 279-289.
(12) Wang, Y.; Hong, W.; Jian, C.; He, X.; Cai, Q.; Liu, W. The Intrinsic Hydrogen Evolution Performance of 2D Molybdenum Carbide. J. Mater. Chem. A 2020, 8 (45), 24204-24211.
(13) Tan, S. M.; Ambrosi, A.; Sofer, Z.; Huber, Š.; Sedmidubský, D.; Pumera, M. Pristine Basal- and Edge-Plane-Oriented Molybdenite MoS2 Exhibiting Highly Anisotropic Properties. Chem.—A Eur. J. 2015, 21 (19), 7170-7178.
(14) Xia, Y.; Mathis, T. S.; Zhao, M.; Anasori, B.; Dang, A.; Zhou, Z.; Cho, H.; Gogotsi, Y.; Yang, S. Thickness-Independent Capacitance of Vertically Aligned Liquid-Crystalline MXenes. Nature 2018, 6 (557), 409-412.
(15) Ward, E. J.; Lacey, J.; Crua, C.; Dymond, M. K.; Maleski, K.; Hantanasirisakul, K.; Gogotsi, Y.; Sandeman, S. 2D Titanium Carbide (Ti₃C₂Tₓ) in Accommodating Intraocular Lens Design. Adv. Funct. Mater. 2020, 30, 2000841.
(16) Sarycheva, A.; Polemi, A.; Liu, Y.; Dandekar, K.; Anasori, B.; Gogotsi, Y. 2D Titanium Carbide (MXene) for wireless communication. Sci. Adv. 2018, 4, eaau0920.
(17) Zhang, J.; Kong, N.; Uzun, S.; Levitt, A.; Seyedin, S.; Lynch, P. A.; Qin, S.; Han, M.; Yang, W.; Liu, J.; Wang, X.; Gogotsi, Y.; Razal, J. M. Scalable Manufacturing of Free-Standing, Strong Ti₃C₂Tₓ MXene Films with Outstanding Conductivity. Adv. Mater. 2020, 32 (23).
(18) Li, W.; Weng, C.; Yang, W.; Shen, L.; Bao, N. High-Strength Reduced Graphene Oxide Paper Prepared by a Simple and Efficient Method. J. Mater. Sci. 2021, 56 (25), 14084-14095.
(19) Ling, Z.; Ren, C. E.; Zhao, M. Q.; Yang, J.; Giammarco, J. M.; Qiu, J.; Barsoum, M. W.; Gogotsi, Y. Flexible and Conductive MXene Films and Nanocomposites with High Capacitance. Proc. Natl. Acad. Sci. U.S.A 2014, 111 (47), 16676-16681.
(20) Shekhirev, M.; Shuck, C. E.; Sarycheva, A.; Gogotsi, Y. Characterization of MXenes at Every Step, from Their Precursors to Single Flakes and Assembled Films. Prog. Mater. Sci. 2021, 120, 100757.
(21) Shekhirev, M.; Busa, J.; Shuck, C. E.; Torres, A.; Bagheri, S.; Sinitskii, A.; Gogotsi, Y. Ultralarge Flakes of Ti₃C₂Tₓ MXene via Soft Delamination. ACS Nano 2022, 16 (9), 13695-13703.
(22) Bard, A. J.; Faulkner, L. R. Electrochemical Methods-Fundamentals and Applications, 2nd ed.; 2001.
(23) Zhang, J.; Kong, N.; Uzun, S.; Levitt, A.; Seyedin, S.; Lynch, P. A.; Qin, S.; Han, M.; Yang, W.; Liu, J.; Wang, X.; Gogotsi, Y.; Razal, J. M. Scalable Manufacturing of Free-Standing, Strong Ti₃C₂Tₓ MXene Films with Outstanding Conductivity. Adv. Mater. 2020, 32 (23), 2001093.
(24) Zhang, C. J.; Kremer, M. P.; Seral-Ascaso, A.; Park, S. H.; McEvoy, N.; Anasori, B.; Gogotsi, Y.; Nicolosi, V. Stamping of Flexible, Coplanar Micro-Supercapacitors Using MXene Inks. Advanced Functional Materials. 2018, 28 (9), 1705506.
(25) Lipatov, A.; Lu, H.; Alhabeb, M.; Anasori, B.; Gruverman, A.; Gogotsi, Y.; Sinitskii, A. Elastic Properties of 2D Ti₃C₂Tₓ MXene Monolayers and Bilayers. Sci. Adv. 2018, 4 (6), 1-8, eaat0491.
(26) Zhang, C. J.; Anasori, B.; Seral-Ascaso, A.; Park, S. H.; McEvoy, N.; Shmeliov, A.; Duesberg, G. S.; Coleman, J. N.; Gogotsi, Y.; Nicolosi, V. Transparent, Flexible, and Conductive 2D Titanium Carbide (MXene) Films with High Volumetric Capacitance. Adv. Mater. 2017, 29 (36), 1702678.
(27) Maleski, K.; Shuck, C. E.; Fafarman, A. T.; Gogotsi, Y. The Broad Chromatic Range of Two-Dimensional Transition Metal Carbides. Adv. Opt. Mater. 2020, 9, 2001563.
(28) Lioi, D. B.; Joshua Kennedy, W.; Stevenson, P. R.; Seymour, B. T.; Neher, G.; Schaller, R. D.; Gosztola, D. J.; Vaia, R. A.; Vernon, J. P. Simultaneous Ultrafast Transmission and Reflection of Nanometer-Thick Ti₃C₂Tₓ MXene Films in the Visible and near-Infrared: Implications for Energy Storage, Electromagnetic Shielding, and Laser Systems. ACS Appl. Nano Mater. 2020, 3 (10), 9604-9609.
(29) Hantanasirisakul, K.; Zhao, M. Q.; Urbankowski, P.; Halim, J.; Anasori, B.; Kota, S.; Barsoum, M. W.; Gogotsi, Y. Fabrication of Ti₃C₂Tₓ MXene Transparent Thin Films with Tunable Optoelectronic Properties. Adv. Electron. Mater. 2016, 2, 1600050.
(30) Shuck, C. E.; Ventura-Martinez, K.; Goad, A.; Uzun, S.; Shekhirev, M.; Gogotsi, Y. Safe Synthesis of MAX and MXene: Guidelines to Reduce Risk During Synthesis. ACS Chemical Health & Safety. 2021, 28 (5), 326-338.

### Additional Disclosure

Biological signal transduction and cellular communication rely on a plethora of electrical, electrochemical, and chemical processes.¹ Disruptions in these pathways can directly result in physiological disorders. Bidirectional interfaces with tunable electrical, chemical, microfluidic, and optical properties have enabled investigations into and modulation of complex pathways.^{2,3}

Conventional bioelectronic interfaces rely on highly conductive noble metals, such as Au and Pt, and semiconductors (Si). However, though these electrodes (e.g., Utah array) have achieved small dimensions and dense electrode packing, they are expensive, rigid, and have a limited surface area and high impedance, leading to a lower signal-to-noise ratio (SNR) and charge transfer properties for stimulation.^{4,5} Numerous materials have been investigated for developing bidirectional bioelectronic interfaces, most notably carbon based (e.g., carbon fiber,⁶ CNT,⁷ graphene⁸) and conductive polymers.⁹ Carbon-based electrodes are soft, lightweight, and—in addition to recording and stimulation—can be used for sensing neurotransmitters like dopamine.¹⁰⁻¹² Carbon fibers, however, are brittle: they require a millimeter-scale shell for support during electrode insertion, which limits penetration depth and increases the overall device footprint.¹³ They also lack electrocatalytic capabilities that limit the sensitivity for detecting certain chemical species like hydrogen peroxide (H₂O₂), which is important in studying degenerative diseases like Alzheimer's and Parkinson's.¹⁴ Conductive polymers such as poly(pyrrole) (PPy), poly(aniline) (PANI), and poly(3,4-ethylene dioxythiophene) (PEDOT) face impediments due to trade-offs between electrical conductivity and processability, and issues regarding long-term stability and delamination.¹⁵ Therefore, it is important to identify materials with high conductivity, biocompatibility, and the required mechanical properties.

MXenes can address the aforementioned material and processing challenges. MXenes are a large family of layered two-dimensional (2D) transition metal carbides, nitrides, and carbon nitrides, offering the highest electrical conductivity among solution-processable 2D materials.^{16,17} The most optimized MXene, Ti₃C₂Tₓ, can achieve an electrical conductivity of over 20 000 S/cm, approximately 5-10 times higher than reduced graphene oxide (rGO).¹⁸ Their 2D morphology, easy processability, and hydrophilic functional-group-decorated surfaces make them ideal coating materials for various substrates, offering functional enhancements with minimal increase in weight. Since their discovery at Drexel University in 2011, MXenes have achieved repeatable kilogram-scale productions and have experienced rapid growth across various fields, particularly in biomedical applications.¹⁹⁻²¹ These span from electrophysiology, biosensing, tissue engineering, and therapeutics to antibacterial and antiviral applications.²²⁻²⁴ Specifically, Ti₃C₂Tₓ MXene is biocompatible and does not exhibit cytotoxicity to a wide variety of mammalian cells and tissues.²⁵⁻²⁹ It has enabled implantable and wearable bioelectronics for invasive and noninvasive recording of animal and human electrophysiology.^{27,30-32} Ti₃C₂Tₓ MXene microelectrodes displayed a 4-fold lower impedance and greater capacitance than gold electrodes, leading to enhanced electrophysiological sensing capabilities with a higher SNR.²⁷ Moreover, MXene electrodes can be conveniently sterilized using conventional methods without chemical and performance degradation.^{3.3}

Despite the impressive performance demonstrated shown by earlier MXene electrodes, challenges persist in tissue insertion due to their planar and thin geometry, limited versatility issues due to prefixed designs, suboptimal MXene conductivity caused by the misalignment of MXene flakes, and the time- and resource-intensive nature of microfabrication. Electrical conductivity aside, Ti₃C₂Tₓ MXene is highly sensitive to H₂O₂, as it exhibits a low reduction onset potential due to its electrocatalytic capabilities. 34 However, this capability has not been demonstrated without a bulky current collector or within biological systems.³⁵ Therefore, the demand exists for miniaturized MXene electrodes with simplified fabrication, high electrical conductivity, and integrated capabilities for electrical and chemical sensing, as well as modulation.

Compared to electrodes of other geometries, fiber-shaped electrodes provide higher spatial and temporal resolution because of their proximity to target biological cells.³⁶ A small electrode diameter (D) decreases insertion resistance and associated damage. It significantly reduces the bending stiffness K∝D⁴, resulting in less tissue damage and signal loss-vital for long-term implants.³⁷ Furthermore, unlike planar electrodes with fixed device configurations, microfiber electrodes offer greater flexibility and fewer geometric constraints. They can be inserted into or wrapped around target tissues of varying geometries, even positioned between cells as individual electrodes or as a bundle.³⁸⁻⁴⁰ This versatility makes them applicable to various scenarios. Recently, thermal drawing has emerged as a powerful method for fabricating versatile neural probes.^{41.42} However, this process imposes limitations on material selection and faces challenges in reducing the diameter below 100 μm. Additionally, the slow production speed makes thermal drawing challenging for meeting the large-scale and multimodal requirements of biointerfaces. The easy processability and multifunctionality of MXenes provide an alternative, streamlined process for making high-performance, fiber-shaped electrodes.

Here, we propose a rapid, scalable, and versatile dip-coating technique for manufacturing electrically conductive and flexible Ti₃C₂Tₓ microfiber electrodes that can be easily customized for various biological studies. This method produces MXene-coated fibers with tunable mechanical, electrical, and electrochemical properties, featuring uniform MXene coatings that are precise and reproducible. These qualities were unattainable in previous MXene fiber coating studies.^{43.44} Moreover, we utilized the shear force in the MXene solution meniscus during dip coating to align and conformally coat MXene flakes along the surface of individual nylon filaments, a strategy that has not yet been utilized in the coating of MXene fibers. This achieved low linear resistance (as low as 9.3±1.1 Ω/cm) even at high drawing speeds (up to 15 mm/s). These MXene-coated filaments can be efficiently batch-fabricated into arrays of fiber electrodes, encapsulated with an insulating layer of Parylene C, and exposed only at the tip upon application for electrical recording, stimulation, and H₂O₂ sensing. The MXene fiber microelectrodes exhibited excellent knotting and favorable mechanical properties for handling and implantation. We demonstrated the versatility of these simply made, multifunctional fiber electrodes both in vivo in rat nerves and muscles and ex vivo in bladder tissue. These fiber microelectrodes provide an economical, durable, and user-friendly miniaturized platform for various biological applications.

### Results and Discussion

Although the following examples comprise MXene and parylene coatings placed on nylon fibers, it should be understood that these examples are illustrative only and are not limited to these particular fibers and coatings.

### MXene-Enabled Microfiber Electrode Fabrication.

In this study, we leveraged the advantageous characteristics of MXenes and nylon to fabricate flexible, fiber-shaped microelectrodes with dip coating. Nylon filaments were chosen as substrates due to their lightweight, high chemical resistance, mechanical durability, and cost-effectiveness.⁴⁵ Moreover, nylon is a U.S. Food and Drug Administration (FDA) approved material for medical devices such as permanent sutures, catheters, and dental implants.^{46.47} The nylon fibers are produced through melt spinning in a continuous manner. The positive charge on nylon leads to strong binding of the negatively charged MXene.⁴³ If a continuous MXene coating is applied, it can enable electron conduction along the entire length of the filament. Round nylon filaments of varying diameters are chosen for this proof-of-concept study to simplify MXene/substrate interactions in the dynamic meniscus and ensure comparability with current fiber microelectrode probes in bioelectronics.

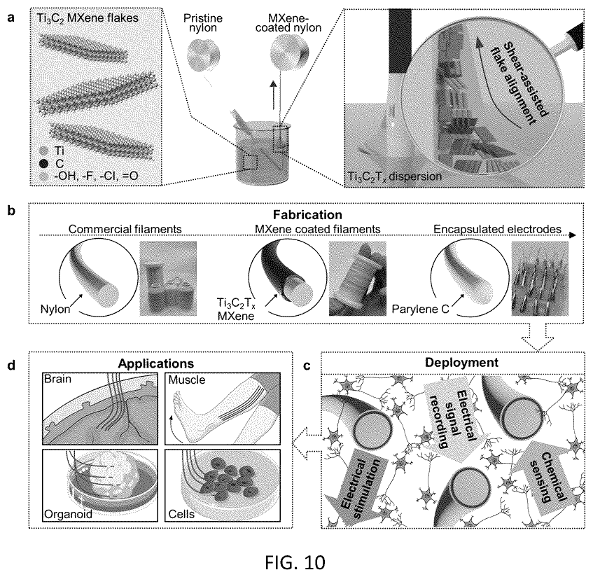

Single-layer Ti₃C₂Tₓ flakes, with an average flake size of 1 μm, were synthesized through a mixed-acid method. The successful synthesis was confirmed using UV-vis spectroscopy and X-ray diffraction (XRD) analysis.^{20,48} The abundance of negatively charged functional groups (e.g., —F, —Cl, OH) on Ti₃C₂Tₓ flakes lead to a large negative ζ-potential in water (−50 mV), allowing them to form a stable solution for dip coating without any surfactants or additives. Furthermore, the solution rheology can be tuned over a wide range by adjusting MXene flake size and/or concentration, eliminating the necessity for rheological thinners and thickeners. The simple coating formulation of MXene in water is preferable for implantable electrodes when additives may complicate the biological response. In contrast to graphene oxide (GO), MXene does not require thermal or chemical reduction to achieve high conductivity. The MXene dip coating process is illustrated in FIG. 10a: the nylon filament is immersed in the MXene solution and vertically drawn. A stable meniscus forms thanks to the favorable electrostatic interactions between nylon and MXene. This enables a consistent layer of MXene dispersion to adhere to the fiber, where MXene flakes are aligned by shear force. This layer dries into a MXene coating at a uniform thickness.

MXene-coated filaments can be selected based on their properties, cut into desired lengths for intended applications, and encapsulated with a 10 μm thick layer of Parylene C (FIG. 10b). Parylene is a United States Pharmacopeia (USP) Class VI polymer that is widely used as a biocompatible encapsulation for chronic medical devices.^{49.50} It has become a popular substrate and passivation layer for bioelectronics due to its barrier properties, flexibility, and processability.⁴⁹ Once the encapsulated fiber is cut at the tip, a controllable amount of MXene is exposed, providing the benefits of easy handling and reproducible outcomes (FIG. 10c). This represents a significantly simplified electrode fabrication and deployment process compared to microfiber electrodes made from other materials, e.g., brittle carbon fibers that require extensive polishing.^{37,51,52} These electrodes can be produced to different lengths to investigate biological systems at various scales, ranging from cells (˜10-20 μm), organoids (˜1-5 mm), and tissues (>1 cm) to the brain and muscles, in vivo, in vitro or ex vivo (FIG. 10d).

### Tailoring Electrode Properties Through Dip Coating Parameters.

Although dip coating is a widely used technique for applying MXenes to various substrates like glass, PET, foams, yarns, and fabrics owing to its simplicity and control, this method has yet to be demonstrated in a continuous fashion on a regularly shaped single filament.^{18,53} Fluid dynamics theories, most notably the Landau-Levich-Derjaguin (LLD) model, have been developed to describe the flow dynamics within the meniscus and bulk solution when a fiber is vertically withdrawn from the surface of a Newtonian liquid. This process involves an equilibrium between the capillary action induced by substrate drawing and the viscous drag exerted by the liquid.⁵⁴ The coating thickness (h_{fiber}) on fine fibers can be approximated as follows

$${h}_{fiber} = {A}_{fiber} C {a}^{2 / 3} R \qquad ( 1 )$$

where A_{fiber} is 1.34, a unitless value representing the effect of the fiber geometry on the interface; R is the fiber radius; capillary number (Ca) is defined as Uη/σ, where U is the drawing speed, η is viscosity and σ is the liquid's surface tension. Thus, an increase in drawing speed, viscosity, and fiber diameter should increase coating thickness. However, the model becomes considerably more complicated for non-Newtonian fluids. MXenes suspensions exhibit shear thinning behavior, where their viscosity decreases with an increase in shear force, the extent of which depends on the size distribution of MXene flakes and their concentrations.^{55,56}

Additionally, due to their large aspect ratio, MXenes can spontaneously organize into a liquid crystalline state above a critical transition concentration, which can be calculated for a given suspension based on the MXene flake size and concentration.⁵⁷ The liquid-crystalline state of MXenes has been to facilitate flake alignment during wet spinning or blade coating, where shear force is present.^{57,58} Consequently, during dip coating, the shear force distribution gradients in the meniscus will be influencing and be influenced by the local viscosity and alignment of MXene in solution, making the theoretical prediction of MXene coating thickness challenging.⁵⁹ Thus, we designed an experimental approach to investigate the impact of three parameters—namely MXene concentration, filament diameter, and drawing speed—on coating thickness and resulting properties.

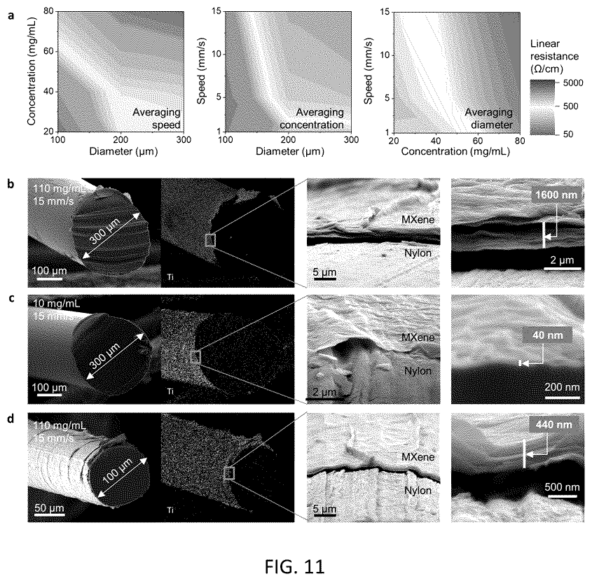

A parametric study was conducted across three levels for each parameter: 20, 40, and 80 mg/mL for MXene concentration; 100, 200, and 300 μm for nylon filament diameter; and 1, 5, and 15 mm/s for coating speed. The maximum speed allowed for the dip coating setup is 15 mm/s. Increasing the MXene concentration significantly raises the viscosity of the solution, transforming it from a liquid resembling water to a paste, despite shear thinning being observed across all employed MXene concentrations. Consistent and uniform coatings were achieved on all 27 conditions (n=5 samples per condition). The resistance measurements of the coated filaments were summarized (FIG. 11a). As MXene concentration, filament diameter, and drawing speed increased, the resistance decreased by more than 2 orders of magnitude, from over 5 k Ω/cm to below 40 Ω/cm. This aligns with the trends predicted by eq 1, indicating the presence of comparable fluidic dynamic mechanisms within the explored parameter space. Furthermore, standard deviation (SD) decreased with average resistance, indicating a more uniform coverage and enhanced electron percolation network with the accumulation of MXene flakes.

Additionally, to ascertain the relative impact of each parameter, we presented the mean resistance for each parameter in a main factor analysis fashion, which revealed that MXene concentration and filament diameter exert a more substantial influence. Therefore, the range of MXene concentration and filament diameter was further expanded by adding concentrations of 10 and 110 mg/mL and filaments with a diameter of 28 μm. A concentration of 110 mg/mL was the highest concentration we achieved through a centrifugation-based concentration method. The trend of decreasing resistance with increasing concentration persisted, with the 110 mg/mL MXene solution yielding the lowest linear resistance in this study, measured on the 300 μm filament. Similarly, the trend of increasing resistance with decreasing diameter continued for 28 μm filaments, producing a coating with a resistivity of ˜600 Ω/cm, when the 110 mg/mL MXene solution was used.

To gain insight into the coating mechanism, we utilized scanning electron microscopy (SEM) imaging to examine the MXene coatings on specific filaments (FIGS. 11b-d). The energy dispersive spectroscopy (EDS) confirmed the conformal MXene coatings on the nylon fibers. For a 300 μm-diameter filament, the 110 mg/mL MXene solution produced a thick coating, measuring approximately 1.6 μm in thickness, 20 μg/cm in weight with a metallic purple hue and a slightly wrinkled morphology characteristic of Ti₃C₂Tₓ. In contrast, the 10 mg/mL MXene solution resulted in an ultrathin coating of approximately 40 nm with negligible weight increase. The 11 times difference in MXene concentrations brought a 40 times difference in coating thickness. Based on eq 1, the disproportional difference can be attributed to the significantly higher viscosity of the 110 mg/mL dispersion than 10 mg/mL. The thickness of the 110 mg/mL coating, measuring 1.6 μm, is notable, given that low MXene concentrations (1-30 mg/mL) are typically recommended and employed in dip coating.^{43,44,60-63} These dilute MXene solutions lead to thin MXene coatings with high resistances, requiring the repetition of dip coating cycles to achieve a thicker deposition. In our study, we demonstrated that viscous MXene solutions, reaching up to 110 mg/mL, are suitable for dip coating when assisted by the high shear force generated from fast coating speed. This enabled the rapid and continuous production of highly conductive filaments in a single pass, with low MXene consumption (<20 μg/cm) resulting in minimal coating costs. Without the use of temperature evaluation and specialized equipment, and the accompanying substrate material limits, as in the case of thermal drawing,⁶⁴ this method is more cost-effective and accessible for production purposes. On the effect of filament diameter, at the same concentration of the 110 mg/mL solution, the filament of a smaller 100 μm diameter received a thinner coating of 440 nm, as anticipated from eq 1.

When examining resistance in conjunction with coating thickness, an intriguing observation emerges. The 40-fold difference in coating thickness between 110 and 10 mg/mL of MXene on 300 μm diameter filaments resulted in a 73-fold decrease in resistance. If we approximate MXene coating as a free-standing film and calculate electrical conductivity, the coating from the 110 mg/mL MXene solution should have a conductivity of 7093±819 S/cm, while the coating from the 10 mg/mL MXene solution should have a conductivity value of 3891±249 S/cm. The former approaches the electrical conductivity of 8875±412 S/cm measured for a free-standing film created by vacuum-assisted filtering of the same Ti₃C₂Tₓ solution. We attribute the higher conductivity from 110 mg/mL to its liquid crystalline ordering and its resulting higher order of flake alignment in the presence of shear force in the meniscus, which is confirmed by rocking curve XRD analyses of the (002) peaks of the coatings. This higher order of alignment allows the 110 mg/mL MXene-coated filaments to demonstrate not only lower resistances than commercial silver-plated nylon filaments but also at a lower amount (wt %) of active material. For example, 100 μm nylon filaments coated with 110 mg/mL MXene exhibit a resistance of 41.9±6.1 Ω/cm at 5.3 wt % active material loading, contrasting with the 200 Ω/cm resistance at a 10.0 wt % for silver-plated nylon of the same diameter. Low linear resistance is critical to the performance of fiber-shaped electrodes, as the overall electrode resistance scales linearly with fiber length. It implies that MXene fiber electrodes do not require a current collector, eliminating the need for additional steps such as gold deposition⁶⁵ and metal wire attachment⁶⁶ that are typically required to enhance the electrical conductivity of rGO electrodes. A current collector can, however, be present.

We then calculated the bending stiffness of MXene-coated nylon electrodes, a critical property influencing the mechanical compatibility of the electrodes with tissues.³⁶ Rigid electrodes are less compatible with soft tissues since they trigger increased stiffness-related foreign body response.⁶⁷ The subsequent immune response and device encapsulation reduce the recording and stimulation capabilities. Thus, the lower stiffness of MXene-coated and Parylene-encapsulated electrodes compared to other conductive fibers that have been considered as neural probes, such as carbon fiber and fibers made of CNT, platinum, tungsten, and silicon at comparable diameters, is beneficial. This suggests that MXene electrodes can possess greater mechanical compatibility with biological tissues, such as brains and muscles. Despite their lower stiffness, their fiber geometry allowed direct, assistance-free insertion of all fibers (28-300 μm diameter) directly into deep tissue, modeled with 0.6 wt % agarose gel—a composition commonly employed to mimic the mechanical properties of the brain.

By varying processing parameters, we demonstrated dip coating as a facile method for producing conductive filaments with tunable MXene coating thickness, mechanical and electrical properties. For the subsequent electrochemical characterizations, meters of coated filaments with varying parameters were produced quickly and cut into 5 cm-long electrodes and then encapsulated with Parylene C. Thanks to the low resistance from aligned MXene flakes, these electrodes can be made into longer lengths for deep brain or muscle stimulation in large animals (e.g., nonhuman primates⁶⁸) without concerns about signal loss associated with a significantly higher resistance. These MXene electrodes can be easily “activated” for deployment and shortened, if necessary, by cutting them with a fresh razor blade against a hard surface (e.g., a glass substrate) at room temperature. This method is adequate for producing regular and consistent cross sections for electrochemical characterization, which was performed to assess the performance, reliability, and stability of the MXene electrodes.

### Electrochemical Characterization of MXene Electrodes.

While low impedance and high capacitance are typically desirable for effective sensing and stimulation, the specific physical, mechanical, and electrochemical requirements for electrodes vary depending on the targets and goals. For example, clinical deep brain stimulation platforms necessitate electrode geometries with a high aspect ratio.⁶⁹ In contrast, transcutaneous electrical nerve stimulation requires larger electrodes capable of delivering higher magnitudes of stimulation currents.⁷⁰ This renders our method especially valuable for numerous biological applications. To ensure proper electrode selection and safe operation, it is essential to conduct electrochemical characterizations on MXene dip-coated electrodes produced under various processing parameters. In addition, to ensure the success of electrode application, the electrodes should exhibit high reliability and stability. Cyclic voltammetry (CV) and electrochemical impedance spectroscopy (EIS) tests were conducted to provide insights into the capacitance and impedance of MXene electrodes in relation to both MXene concentration and filament diameter in a three-electrode setup.

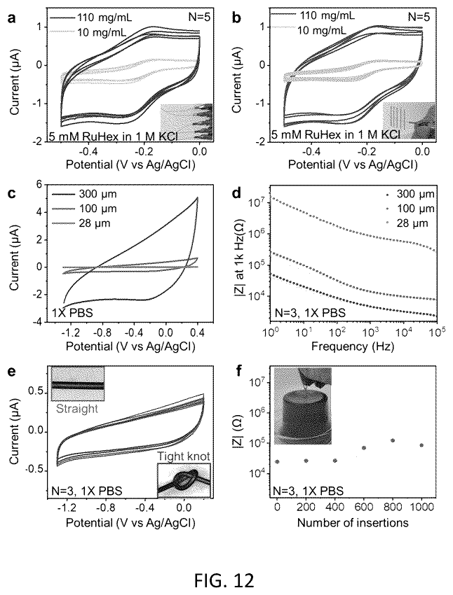

First, 10 electrodes of 300 μm diameter, 5 coated with 10 mg/mL and the other 5 coated with 110 mg/mL MXene, were characterized with CV in 5 mM hexamine-ruthenium (III) [Ru(NH³)⁶]³⁺ (RuHex) in 1 M KCl at 20 mV/s (FIG. 12a). RuHex, a standard outer-sphere redox probe, was chosen as the electrolyte due to its sensitivity to surface area (not sensitive to surface chemistry). The high degree of CV overlap among different electrodes indicates a consistent and uniform coating. A higher capacitance was observed when comparing electrodes coated with a concentration of 110 mg/mL to those coated with 10 mg/mL. This is attributed to the thicker coating resulting from the higher concentration of MXene in the 110 mg/mL solution. For the same reason, lower impedance was observed in the 110 mg/mL electrodes compared to the 10 mg/mL electrodes. Moreover, the oxidation and reduction of RuHex occurred at a centered potential of −0.2 V (vs Ag/AgCl), as expected.⁷¹ The semisigmoidal CV shape suggests minimal diffusion limitations, which can improve speed and sensitivity in analyte detection. Next, one electrode was chosen from each of the 110 and 10 mg/mL sets. A CV was recorded after an initial cut, and then the incision was retracted by 1-2 mm, exposing a fresh cross-section of the same fiber, followed by recording another CV. This process was repeated for a total of five iterations (FIG. 12b). Consistent high degrees of overlapping CVs both support the uniformity of the coating and also underscore the reliability of the electrode-cutting method. This method consistently exposes uniform cross sections. The effectiveness of the Parylene C insulating layer is evident from the significantly higher capacitance and lower impedance observed in nonencapsulated electrodes compared to the encapsulated ones.

To understand the relationship between fiber diameter, capacitance, and impedance, we selected 110 mg/mL MXene-coated electrodes with diameters ranging from 300 to 28 μm. We employed 1× phosphate-buffered saline (PBS), a simplified representation of human body fluids commonly used in electrochemical characterizations of recording and stimulation electrodes. This choice allowed for a meaningful comparison between MXene electrodes and electrodes made from other materials. The safe operational window for MXene fiber electrodes in PBS, determined at a scan rate of 100 mV/s, ranges from −1.3 to 0.4 V, with 28 μm electrodes exhibiting a slightly earlier onset of oxygen evolution reaction. As the diameter decreased from 300 to 28 μm, a notable reduction in capacitance was observed due to the thinner coating and smaller exposed MXene ring at the electrode tips (FIG. 12c). The cathodic charge storage capacity (CSCc) was determined from CV scans by integrating the cathodic current over time and normalizing it by both the entire cross-sectional area and the active material area, providing indicators for the sensing performance of the electrode and the active material (MXene), respectively. Larger CSCc values indicate higher sensitivity to electrical signals.

When compared with electrodes of other materials such as carbon fiber, graphene fibers, CNT, SI, PEDOT: PSS, and Pt, MXene electrodes exhibited significantly higher CSCc when normalized by the active area of the coating. The heightened performance is likely attributed to two factors: the advantageous diffusion in microelectrodes due to the reduction in electrode diameter and coating thickness, and the increased surface area resulting from the 2D stacked morphology of MXene, facilitating capillary actions when the edges are exposed. This observation aligns with the results of a recent study that compared 2 μm thick MXene thin film electrodes with edge-exposed and basal-exposed configurations.⁷¹ These factors also provided the 300 and 100 μm electrodes with low impedances of 4.70±0.55 and 14.0±3.1 kΩ, respectively, at 1 KHz-among the lowest reported for implantable microfiber electrodes (FIG. 12d). The phase responses from EIS are typical for smooth, compact electrodes, consistent with the SEM and XRD findings for the MXene electrodes.⁷² Finally, we observed an increase in impedance and variations with decreasing fiber diameter due to the reduction in MXene surface area available for charge exchange, presenting challenges in recording high-quality electrical signals with 28 μm thin electrodes.

Furthermore, the MXene coating, nestled between the substrate polymer and the encapsulating layer of Parylene C, exhibited exceptional knotting performance. Identical CVs were recorded before and after tying knots as tightly as possible, representing a more mechanically demanding test than bending and knotting with less curvature (FIG. 12c). Electrode durability to insertion was also evaluated. Only a slight change in impedance was observed after 1000 repeated insertions, with negligible alteration in impedance and CV (FIG. 12f). Furthermore, while achieving long cyclability proved challenging within the extended window of −1.3 to 0.4 V in 1×PBS, reducing the window to −1.1 to 0.3 V obtained excellent cyclability of sustained performance over 5000 cycles at 100 mV/s by avoiding hydrogen and oxygen evolution reactions near the cathodic and anodic turnover potentials. Only minimal capacitance and impedance changes were observed even after 12 months of bench storage. This can be attributed to Parylene effectively protecting MXene against moisture in the air, a factor known to induce hydrolysis and oxidation of MXene, but also to the high stability of Ti₃C₂Tₓ once it is processed into a film.⁷³ When the 12 months electrode was immersed 35 mm versus 2 mm depth into 1×PBS, a consistent performance was observed, allowing for reliable readings from different depths within a tissue. Collectively, all tests demonstrate the versatility of the MXene dip coating process in reliably producing robust, fiber-shaped electrodes with varying capacitance and impedance.

Ex Vivo Sensing of H₂O₂ in Murine Bladder Urothelium.

Reactive oxygen species (ROS) are essential for cellular processes, including signaling, immune responses, and redox regulation. Their irregularities induce oxidative stress, causing significant biological damage, leading to chronic diseases, acute conditions, and aging.⁷⁴ One of the most important ROS is H₂O₂, the fluctuation of which serves as an indicator for Alzheimer's and Parkinson's diseases. 35 Thus, measuring and monitoring H₂O₂ is key for monitoring oxidative stress and assessing the impact of ROS in cellular and physiological processes.

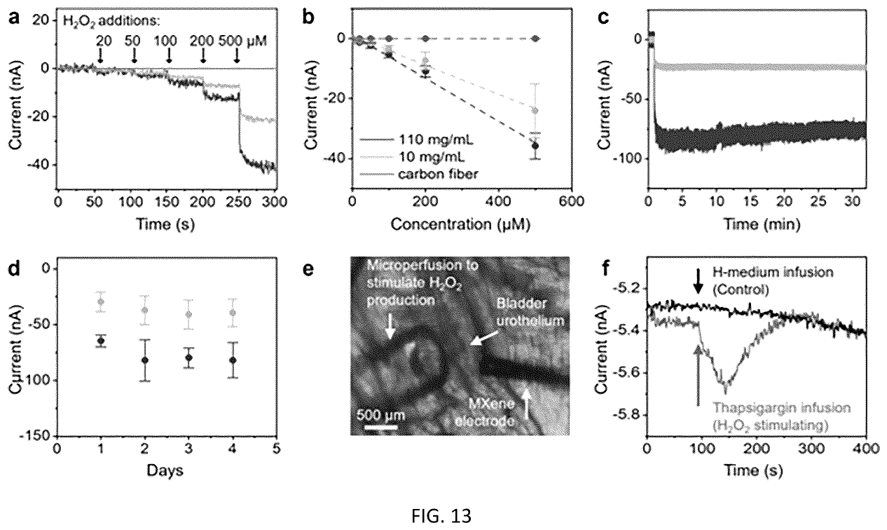

We employed chronoamperometry to reduce H₂O₂ at −650 mV (vs Ag/AgCl), considering the limited positive potential window for H₂O₂ oxidation determined in FIG. 12c, for two types of MXene electrodes: 300 μm electrodes coated with 10 and 110 mg/mL MXene, respectively. H₂O₂ concentrations (20, 50, 100, 200, and 500 μM) were sequentially added every 50 s to 10×PBS (FIG. 13a). The selection of these two types of electrodes allowed us to understand the effect of MXene coating thickness (exposed area of MXene at the tip) on the sensitivity of H₂O₂ detection. We observed a good linear response in current for both 10 mg/ml (r²=0.810) and 110 mg/ml (r²=0.965) MXene-coated electrodes across the entire concentration range (FIG. 13b). While the slope of the line, representing the sensitivity of the whole electrode, was higher for 110 mg/mL, normalization by the cross-sectional MXene area demonstrates that the 10 mg/mL electrode had a higher sensitivity (−148.39 μA/μM/cm²) compared to the sensitivity of 110 mg/mL electrode (−4.73 μA/μM/cm²) per unit area of the active material. This is likely attributed to enhanced diffusion resulting from the decrease in coating thickness. The electrodes exhibited excellent stability in continuous runs (lasting 30 min with 1 mM of H₂O₂) and over consecutive days with 1 mM of H₂O₂ (FIG. 13c,d). In comparison, carbon fiber electrodes, while exhibiting the usual electrochemical performance against RuHex showed no response toward H₂O₂ due to the lack of electrocatalytic activity. These results underscore the H₂O₂ detection capability of the MXene-coated electrodes, all without the need for any additional current collector or chemical treatment. This achievement is attributed to the well-aligned flakes during coating and the redox-active surface of MXene flakes.

Next, we utilized a 110 mg/mL MXene-coated electrode to monitor H₂O₂ production in bladder urothelium when stimulated using 200 nM thapsigargin through microperfusion (FIG. 13c). This endeavor led to the successful detection of H₂O₂ amperometrically from bladder urothelium tissue. As shown in FIG. 13f, upon perfusion of 200 nM thapsigargin, a larger current was observed and attributed to the production of H₂O₂. Our study proves the capability of MXene for H₂O₂ sensing in a biological system, enabling the translation of these findings into microfiber applications.

### In Vivo Electrical Recording and Stimulation in Sciatic Nerve.

Electrical stimulation is one of the most prominent clinical techniques for modulating electrophysiological activity. Stimulation is achieved by injecting current pulses at the electrode-tissue interface, which induces depolarization of the cell membrane.⁷² This has enabled therapeutic interventions such as deep brain stimulation for epilepsy and transcutaneous electrical nerve stimulation for pain relief.^{75,76} The electrical stimulation performance of an electrode is typically assessed using chronopotentiometry, where a charge is injected through biphasic, charge-balanced current pulses into the electrode in a 1×PBS electrolyte.^{9,72} A cathodic current pulse results in the generation of a potential transient, with the cathodic excursion potential (E_{c}) recorded 10 us after the cathodic pulse concludes. For safe operation, the maximum charge of an electrode is determined as the charge injected when E_{c} reaches the cathodic limit established by CVs, which is −1.3 V, in the case of MXene electrodes. To facilitate direct comparisons across fiber electrodes of different sizes and materials, cathodic charge injection capacity (CIC) is adopted as a standardized measure. This involves normalizing the maximum cathodic charge by area, either considering the entire cross-sectional area or the active material area.

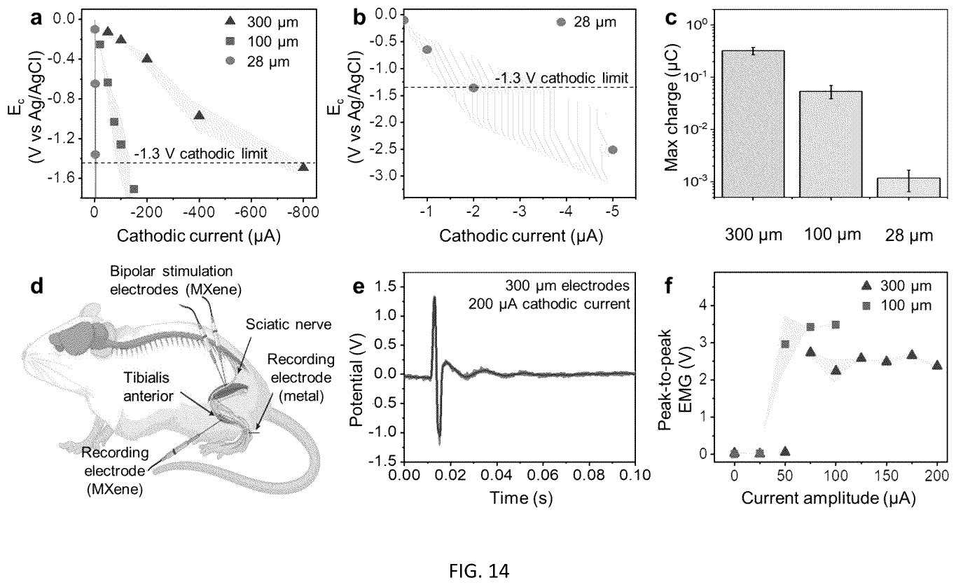

In this study, we conducted chronopotentiometry on MXene electrodes with varying diameters (300, 100, and 28 μm). All electrodes were coated with 110 mg/mL MXene at 15 mm/s, with a set of 3 electrodes being prepared per size. We depicted the relationship between cathodic potential excursion E_{c} and increasing injected cathodic current amplitude, revealing the average E_{c} decrease with the reduction in electrode diameter (FIG. 14a,b). The currents corresponding to E_{mc} at the cathodic limit were calculated as 642.2±101.2, 108.2±30.9, and 2.32±1.05 μA, translating to maximum cathodic charges of 321.1±50.6, 54.1±15.4, and 1.16±0.53 nC, respectively, as the electrode diameter decreased (FIG. 14c). It indicates that thicker electrodes can withstand larger currents, enabling them to deliver more charge to the target tissue or cell if required to elicit a response. Consistent with the established scaling of CIC_{c} with electrode area, the 100 μm electrodes exhibit a higher CIC_{c} of 39.0±11.1 mC/cm² compared to the 300 μm electrodes at 18.8±3.0 mC/cm^{2.31} This is attributed to the fact that smaller electrodes facilitate faster ion diffusion and are less prone to nonuniform current distribution, which is referred to as edge effects. Comparing MXene electrodes with those made of other materials, the CIC^{c} of MXene electrodes is notably higher. This can be attributed to the ring-shaped active area, which again mitigates nonuniform current distribution issues in round cross sections. Additionally, the slits in stacked MXene flakes likely facilitate the wicking of the electrolyte, thereby enlarging the actual surface area in contact with the electrolyte.

We then assessed the performance of our MXene fiber microelectrodes for in vivo electrophysiology studies. Here, we placed a bipolar assembly of our MXene fiber electrodes on the sciatic nerve of a rat for delivering stimulation current pulses (FIG. 14d). An independent MXene fiber electrode was placed on top of the tibialis anterior muscle to record the evoked electromyographic (EMG) activity. Upon application of a stimulating current pulse of 200 μA through a pair of 300 μm MXene electrodes on the sciatic nerve, evoked EMG was successfully recorded (FIG. 14c). The SNR of the evoked EMG, as recorded by the MXene electrodes, was 15.20±0.32 and 13.92±0.53 dB for 100 and 300 μm MXene microfibers, respectively. Increasing the amplitude of the stimulation current on the sciatic nerve led to an increase in the peak-to-peak amplitude of the evoked EMG until maximum recruitment is achieved (FIG. 14c). We note that maximum recruitment was approximately observed with 50 and 75 μA current pulses for 100 and 300 μm MXene fiber electrodes, respectively. This was achieved at current amplitudes much lower than the maximum amplitudes allowed or 100 and 300 μm MXene electrodes (642.2±101.2 and 108.2±30.9 μA, respectively). We benchmarked the stimulation and recording capabilities of the MXene microfibers against commercially available tungsten (W) and stainless-steel electrodes, respectively. Stimulation through the bipolar W electrodes follows a similar trend as that of the MXene electrodes, with maximum recruitment occurring around 80 μA. On the other hand, the evoked EMG recordings via the stainless-steel electrodes exhibited an SNR of 19.72±0.52 dB. The greater SNR of these electrodes can be attributed to their higher conductivity and greater geometric footprint of these electrodes, since the lateral surface of the stainless-steel wires is exposed to the muscle tissue as well. This provides in vivo demonstration of MXene fiber microelectrodes for sensing and modulating electrophysiological activities.

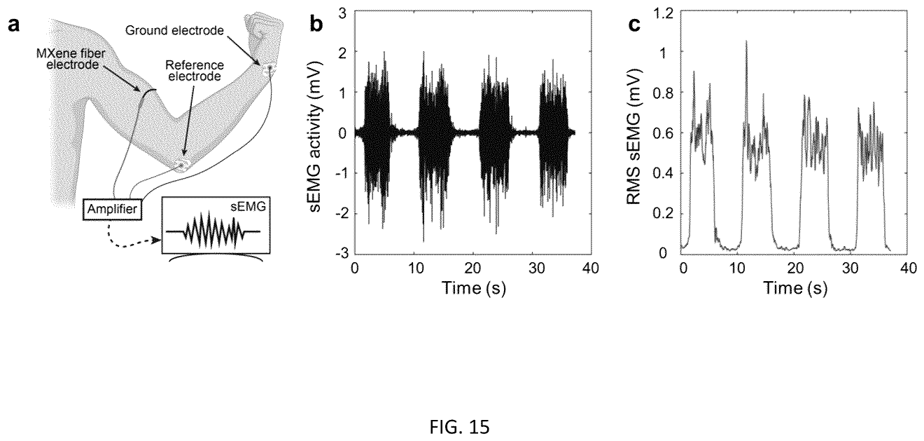

Furthermore, we tested the use of our MXene-coated fiber microelectrodes in wearable and clinical applications. We placed a representative 300 μm diameter MXene-coated nylon fiber (without Parylene C encapsulation, 80 mg/mL MXene coated at 15 mm/s) on the skin over the belly of the biceps u of a healthy volunteer (FIG. 15a). The microelectrodes were able to successfully record sEMG activity during voluntary contraction of the biceps (FIG. 15b,c). This demonstrates the use of the MXene fibers to be integrated into textiles as wearable sensors, further showcasing the versatility of microfiber electrode applications.

Through this study, we demonstrate dip coating as an efficient and versatile method for producing MXene-functionalized filaments with customizable properties. The continuous dip coating process shows promise for seamless integration into nylon filament production, ensuring uninterrupted electron conduction throughout the filament's length. It would be interesting to apply this method to filaments of other materials, such as biodegradable polylactic acid and carbon fibers, as well as filaments of alternative geometries like hollow or grooved shapes. Hollow filaments can add microfluidic functions, while the grooved filaments have more surface for MXene to adhere. Various MXene compositions can be adopted, including V₂CTₓ, which has demonstrated improved performance for ROS sensing, and Ti₃C₂Tₓ/GO, where GO enhances sensitivity to numerous neurotransmitters.^{11,77} More parametric studies will be needed to understand how changes to other fiber substrates and MXene formulations, as well as how substrate chemistry, tension, and geometry interact with MXene flake size, concentration, and composition. These interactions will determine the optimal combination of coating parameters for conductivity and productivity. It is likely that fibers with delicate surface structures are best coated with MXene dispersions of low viscosity to penetrate small gaps and achieve highly conformal coatings. It is essential, in the next step, to construct a new coating setup that enables continuous automated production and accommodates faster drawing speeds. This will allow for quick additional parametric studies and experimental verification of the highest drawing speed for a given substrate and MXene dispersion.^{78,79} This adjustment could result in even thicker deposition, reduced resistance, and increased production speed. Moreover, studying the effect of dip coating cycles on coating thickness and conductivity will be of interest to applications where a larger amount of MXenes will be needed, such as for energy storage applications.

In this study, we focused the proof-of-concept demonstration of these electrodes for electrical stimulation, electrical sensing, and H₂O₂ sensing for biointerfacing. However, these MXene microfiber electrodes can be used beyond these specific applications, wherever flexible, fiber-shaped electrodes and current collectors are required. For instance, we envision the possibility of using a thermally responsive polymeric filament as a substrate for photothermal actuated fibers, harnessing the exceptional photothermal conversion efficiency of MXenes. Furthermore, the functional groups on MXenes serve as effective anchoring points for binding antibodies and drugs, thereby making the electrodes useful as vessels for controlled drug delivery. Due to their high electrical conductivity, these electrodes are promising for long-distance strain monitoring as smart sutures and smart composite reinforcements. While thicker MXene coatings are preferred for certain applications, thin coatings are preferred in other applications, such as gas sensing. Here, increased resistances and more exposed material to the gas result in greater resistance changes. The tunable and versatile nature of these MXene fiber electrodes unlocks a plethora of applications across various domains.

### Conclusions

In this study, we provide an exemplary, high-throughput approach for the swift fabrication of versatile microfiber electrodes using Ti₃C₂Tₓ MXene. The utilization of shear force in the high-speed coating process enables the adoption of viscous MXene dispersions at concentrations of up to 110 mg/mL. Through this method, MXene flakes are effectively aligned to produce coatings with a low resistance of ˜10 Ω/cm (electrical conductivity of 7093±819 S/cm) after a single pass at minimal MXene consumption. This innovative process leads to the development of reliable, durable, and cost-effective Ti₃C₂Tₓ microfiber electrodes that are free of additives and current collectors. Furthermore, our adaptable methodology facilitates the easy customization of the electrode's electrical, mechanical, and electrochemical properties by adjusting MXene concentration, filament diameter, and coating speed. These electrodes exhibit multifunctionality, enabling bidirectional electrical communication and H₂O₂ detection, as validated through in vivo and ex vivo studies. Additionally, the electrodes can be efficiently multiplexed for high-density and multimodal arrays and integrated with other investigative techniques such as MRI and optical stimulation. The electrodes can also be used in wearable electronics, particularly when a fiber-shaped current collector or electrode is desired.

### Methods

MXene Synthesis and Characterization. Ti₃C₂Tₓ was synthesized by etching of the Al layer from the MAX phase precursor Ti₃AlC₂ by using a mixture of hydrofluoric acid (HF) and hydrochloric acid (HCl), followed by delamination with an aqueous solution of lithium chloride (LiCl).²⁰ Subsequent centrifugation yielded a stable aqueous dispersion of polydisperse, single-layer Ti₃C₂Tₓ flakes. The concentration of this dispersion was subsequently adjusted to a range of 10-110 mg/mL, achieved through either dilution or high-speed centrifugation. Dynamic Light Scattering (DLS) with a Nano ZS Zetasizer (Malvern Instruments) was employed to determine the flake size distributions and ζ-potential of the MXene dispersion. UV-vis spectra were also obtained using an Evolution 201 UV-vis spectrophotometer (Thermo Scientific) as proof of MXene synthesis quality. The viscosity of the dispersions was measured using a Discovery HR-3 rheometer (TA Instruments) at room temperature, employing a parallel plate configuration (plate diameter 40 mm). To ascertain the electrical conductivity of the synthesized MXene, the sheet resistance and thickness of a free-standing, vacuum-assisted filtration-obtained film were measured using a four-point probe setup (Jandel) and a micrometer, respectively. X-ray diffraction (XRD) analysis was performed on the same free-standing film as additional evidence of the successful synthesis of Ti₃C₂Tₓ. The analysis was carried out using a SmartLab (Rigaku) with a Cu Kα (λ=0.1542 nm) source and a graphite K_{β} filter, operated at a 40 kV voltage and 15 mA current.

Fabrication of MXene Coated Filaments and Characterization. The nylon monofilaments ranging from 100 to 300 μm in diameter and multifilament nylon yarns were purchased from The Thread Exchange. To further reduce the electrode diameter, we isolated single filaments (28 μm in diameter) from a multifilament nylon yarn. The filament coating process began by threading the loose end of a nylon filament from the spool through a needle tip (gauge 14-15G) and then inserting it into a 2 mL round-bottom polypropylene graduated microcentrifuge tube filled with MXene solution. This tube was securely positioned in the lower grips of a 3382A universal tension machine (Instron) with a load cell of 50N, while the loose end of the nylon filament was clamped by the upper grips. As the upper grips moved at a predetermined speed, a thin layer of MXene coating was deposited onto the filament. The upper grips were allowed to reach their maximum height on the 1 m frame and then stopped to permit the coating to air-dry for 5 min. The morphology of the MXene coating was analyzed using a VK-X1000 optical profilometer (Keyence) and a Apreo 2S Lo Vac scanning electron microscope (Thermal Fisher). For SEM imaging, the samples were sputtered with platinum/palladium at 30 mA for 30 s using a sputter coater 108 auto (Cressington Scientific) to prevent sample charging. Cross sections of the MXene-coated filaments were exposed for SEM imaging by cutting them with a fresh blade against a glass substrate at room temperature. To measure the electrical resistance of the MXene-coated filaments, a two-point probe method was utilized with a hand-held multimeter (Klein Tools). Two small stainless steel flat-mouth alligator clips (5.59 mm mouth opening) were soldered, using 60:40 Tin-Lead (Sn/Pb) solder wire, to the probes on the two test leads that were plugged into the multimeter. The alligator clips secure a nonslip, secure contact between the probe and conductive fiber while allowing for easy accommodation of fibers of different diameters. With the clips positioned 1 cm apart along the coated filament, 10 resistance measurements were taken for each sample at different locations and then averaged. The maximum MXene loading (mg/cm) achievable with a single MXene dip coating was determined by weighing 100 cm of MXene-coated filaments (300 μm, 110 mg/mL, 15 mm/s). The weight of 100 cm of pristine 300 μm filament was subtracted from this, and the result was then divided by 100 cm. Note that electrical resistance was selected as the property to track in the parametric study because it can be measured more quickly and accurately than weight or thickness. MXene loading or weight-based measurements were not inaccurate for thinner coatings of less than 1 μm, even at a long sample length of 100 cm. The electrical conductivity of the MXene coating was determined by approximating the coating as a free-standing film with a length (L) of 1 cm, width (W) equivalent to the perimeter of the nylon filament, and thickness (1) representing the coating thickness. Using this method, length/width ratios (W/L) of 10.6, 15.9, and 31.8 were derived for filaments with diameters of 300, 200, and 100 μm, respectively. The resistivity (ρ) of the coating was calculated by

$$ρ = Ω \frac{A}{L} = Ω \frac{Wt}{L} ,$$

from which the electrical conductivity (σ) was obtained as

$$σ = \frac{1}{ρ} .$$

Rocking curve XRD was performed with the same Miniflex II-Gen. Six (Rigaku) to probe the alignment state of flakes in the coating.

Encapsulated Electrodes Preparation. To make the electrodes, MXene-coated nylon filaments were cut into 5 cm segments and connected to stainless steel alligator clips using silver paste. These electrodes were aligned in parallel and coated with a 10 μm layer of Parylene C using a PDS2010 Parylene coater (Specialty Coating Systems). The MXene coating was exposed for testing by carefully cutting the electrode tip with a fresh blade against a glass substrate. The Parylene C encapsulation thickness was determined to be 10 μm. A 10 μm Parylene C coating provides a consistent amount of exposed MXene in the cross section after cutting.

Bending Stiffness Estimation of the Electrodes. The bending stiffness (K) of the electrodes was computed using a core-double shell cylindrical model with the following equation⁸⁰.

$$K = {E}_{c o r e} \frac{π {d}_{0}^{4}}{64} + {E}_{shell 1} \frac{π {d}_{1}^{4}}{64} [ 1 - {( \frac{{d}_{0}}{{d}_{1}} )}^{4} ] + {E}_{shell 2} \frac{π {d}_{2}^{4}}{64} [ 1 - {( \frac{{d}_{1}}{{d}_{2}} )}^{4} ]$$

where E_{core} is the Young's modulus of nylon, d₀ represents the diameter of the nylon filament, Eₛₕₑₗₗ₁ is the Young's modulus of the MXene coating, d₁ represents the total diameter after MXene coating, Eₛₕₑₗₗ₂ is the Young's modulus of Parylene C, d₂ represents the total diameter after Parylene C encapsulation. We used 2.25, 20.6, and 3.17 GPa for the Young's modulus of nylon, 81 MXene coating,⁵⁸ and Parylene C,⁸² respectively. The results demonstrated that the bending stiffness of the electrodes is primarily dictated by the nylon substrate, with stiffness decreasing as the diameter decreases.

Electrochemical Characterization of the Electrodes. A three-electrode setup was utilized, comprising a working electrode (MXene-coated), a reference electrode as Ag/AgCl (3 M KCl), and a platinum wire as the counter electrode. Two electrolytes were prepared: 5 mM ruthenium hexamine (Sigma-Aldrich) in 1 M KCl and 1×PBS (tablets, Sigma-Aldrich). 1×PBS was prepared by dissolving a tablet in 200 mL of deionized water. This setup was employed for conducting CV and EIS characterizations of MXene electrodes with varied parameters in both electrolytes. The characterizations were carried out using a VMP3 electrochemical workstation (BioLogic). To assess the reproducibility of the electrodes, cyclic voltammograms were recorded for five different MXene-coated fibers, of both coating concentrations of 10 and 110 mg/mL. Furthermore, to confirm the uniformity of MXene coating and electrode performance throughout the fibers, five different cross-sectional cuts were made along the fiber using a surgical blade, and recordings were made for each cross-sectional cut. For knotability assessment, the MXene electrode was manually knotted using tweezers and stretched to ensure a tight knot. For durability during insertion, a material with mechanical properties similar to those of a mammal brain was required. For this purpose, agarose gel was prepared by mixing agarose (Sigma-Aldrich Corporation) with deionized water at a concentration of 0.6% w/w, adding a drop of red food coloring for imaging, microwaving the mixture until boiling, and leaving it at room temperature to gel for at least 2 h. An electrode was inserted repeatedly into the agarose gel 3-4 cm deep, with CV and EIS data collected every 200 insertions.

Assessment of H₂O₂ Sensing Capabilities. Amperometry experiments were conducted for both MXene electrodes and carbon fiber at −0.65 V vs Ag/AgCl with in situ additions of H₂O₂ in a 1×PBS solution. The additions resulted in H₂O₂ concentrations of 20, 50, 100, 200, and 500 μM. These concentrations were then plotted against the current to determine the sensitivity of the electrodes, which was calculated from the slope of the line. To assess measurement stability, the applied potential was held for 30 min after the addition of 1 mM H₂O₂. Additionally, measurements of 1 mM H₂O₂2 were tested amperometrically over 4 consecutive days to further evaluate the stability of electrode performance.

Ex Vivo H₂O₂ Sensing in Murine Bladder Urothelium. All procedures were performed according to the regulations of the United Kingdom Home Office and the Animals (Scientific Procedures) Act, and they were approved by the Animal Welfare and Ethical Review Body at the University of Brighton. Wildtype C₅₇BL/6J male mice were euthanized using CO₂ gas followed by cervical dislocation. The entire bladder was then removed and placed in H medium, with a pH of 7.4 (composition: 145 mM NaCl, 5 mM KCl, 1 mM MgCl₂, 0.8 mM CaCl₂), 10 mM Hepes, and 5 mM glucose). The bladder was cut open longitudinally, pinned into a Sylgard lined flow bath, and constantly preferred with H medium. The MXene fiber electrode was placed on the surface of the bladder and held at −0.65 V vs Ag/AgCl. Hydrogen peroxide production was stimulated using 200 nM thapsigargin through microperfusion.

Voltage Transient Measurements of the Electrodes. Charge injection characterization of the MXene microfiber electrodes was performed via voltage transient measurements in a 3-electrode electrochemical cell using a Gamry R600 potentiostat. MXene microfiber electrodes, an Ag/AgCl electrode, and a carbon electrode were used as the working, reference, and counter electrodes, respectively. Asymmetric biphasic current pulses with incrementally increasing current magnitudes were applied at the working electrode. The cathodic, interpulse, and anodic pulse durations were fixed at 500, 100, and 1000 μs. The cathodic potential excursion was defined as the electrode potential vs Ag/AgCl after the end of the cathodic phase of the current pulse (I_{cat}→0 A). The magnitude of the charge injected was calculated as the time integral of the injected charge per cathodic pulse. The charge injection capacity (CIC) was calculated as the area normalized charge injected at a cathodic excursion potential of −1.3 V vs Ag/AgCl for MXene microfibers.

In Vivo Electrical Recording and Stimulation Using MXene Microfiber Electrodes. All procedures were approved by the Institutional Animal Care and Use Committee at the University of Pennsylvania. Male Sprague-Dawley rats (Charles River Laboratories; 300-330 g; aged 6-8 weeks) were anesthetized with isoflurane (5% induction, 1.5-2% maintenance); the hind leg was shaved and cleaned with betadine solution. Meloxicam (2 mg/kg) and bupivacaine (2 mg/kg) were administered subcutaneously in the scruff of the neck and along the incision, respectively. The sciatic nerve was exposed by separating the gluteal muscle. The exposed sciatic nerve was stimulated via symmetric biphasic current pulses of varying amplitudes (0-200 μA), pulse duration of 100 μs, and frequency of 1 Hz (A-M Systems Isolated Pulse Stimulator Model 2100). Electrical stimulation was applied in a bipolar configuration (commercially available bipolar W electrodes, Rochester Electro-Medical, Lutz, FL; #400900, and paired MXene microfiber electrodes). The evoked electromyography (EMG) activity was recorded subdermally over the tibialis anterior using a bipolar configuration with the primary recording electrode placed over the muscle, the reference electrode placed in the tendon, and the ground electrode placed subcutaneously (50 kHz sampling rate; 100× gain and 1-10 000 Hz band-pass; A-M Systems Microelectrode AC Amplifier Model 1800). For recruitment curves, evoked EMG recordings were obtained by gradually increasing the stimulation current amplitude until the amplitude of the evoked EMG plateaued. For each current amplitude, a train of 10 individual stimulation pulses was applied.

Surface Electromyography Data Acquisition and Analysis. Before placing a MXene coated nylon fiber on a healthy volunteer, the skin over the biceps muscle belly was cleaned with an alcohol wipe and moisturized with a few drops of 1×PBS. A 300 μm diameter MXene-coated fiber electrode (80 mg/mL MXene coated at 15 mm/s) was then placed at the target location with an Ag/AgCl reference and ground electrodes (Natus) placed on the bony part of the elbow and the wrist, respectively. sEMG activity was recorded in a monopolar configuration at 5 kHz using a commercial amplifier (Intan RMS2000, Intan Technologies).

Raw sEMG data was bandpass filtered (20-450 Hz) with a fourth order Butterworth filter, and noise due to 60 Hz and its harmonics was filtered out using iterative notch filters. The root-mean-square (RMS) of the filtered sEMG data was calculated over 200 ms epochs with a 50 ms overlap.

Statistical Analysis. All values with error bars represented means±standard deviations (n≥5) and were analyzed using Excel software and plotted using Originlab software. Main factor and interaction plots were conducted and generated using Minitab software. Electrophysiology data analysis was performed using Matlab. Briefly, digital notch filters were applied to remove noise due to 60 Hz and harmonics. For individual stimulation pulses, the recorded evoked EMG was segmented and averaged. Peak-to-peak EMG was calculated as the difference between the maximum and minimum amplitudes of the evoked EMG.

### REFERENCES

(1) Krauss, G. Biochemistry of Signal Transduction and Regulation, 4th ed.; Wiley-VCH: Weinheim, 2008.
(2) Frank, J. A.; Antonini, M.-J.; Anikeeva, P. Next-Generation Interfaces for Studying Neural Function. Nat. Biotechnol. 2019, 37 (9), 1013-1023.
(3) Rastogi, S. K.; Kalmykov, A.; Johnson, N.; Cohen-Karni, T. Bioelectronics with Nanocarbons. J. Mater. Chem. B 2018, 6 (44), 7159-7178.
(4) Deku, F.; Ghazavi, A.; Cogan, S. F. Neural Interfaces Based on Amorphous Silicon Carbide Ultramicroelectrode Arrays. Bioelectron. Med. 2018, 1 (3), 185-200.
(5) Bac, J. H.; Kim, K.; Han, D.; Chung, T. D. Ultra Compact Nanoporous Platinum Coating Improves Neural Recording. Electroanalysis 2021, 33 (4), 839-844.
(6) Vitale, F.; Summerson, S. R.; Aazhang, B.; Kemere, C.; Pasquali, M. Neural Stimulation and Recording with Bidirectional, Soft Carbon Nanotube Fiber Microelectrodes. ACS Nano 2015, 9 (4), 4465-4474.
(7) Wang, K.; Fishman, H. A.; Dai, H.; Harris, J. S. Neural Stimulation with a Carbon Nanotube Microelectrode Array. Nano Lett. 2006, 6 (9), 2043-2048.
(8) Rastogi, S. K.; Bliley, J.; Shiwarski, D. J.; Raghavan, G.; Feinberg, A. W.; Cohen-Karni, T. Graphene Microelectrode Arrays for Electrical and Optical Measurements of Human Stem Cell-Derived Cardiomyocytes. Cell. Mol. Bioeng. 2018, 11 (5), 407-418.
(9) Garg, R.; Balakrishnan, G.; Rashid, R. B.; Gershanok, S. A.; Roman, D. S.; Wang, Y.; Kouassi, P. C.; Rivnay, J.; Cohen-Karni, T. Graphene and Poly(3,4-Ethylenedioxythiophene)-Polystyrene Sulfonate Hybrid Nanostructures for Input/Output Bioelectronics. ACS Appl. Nano Mater. 2023, 6 (10), 8495-8505.
(10) Meng, S.; Liu, Y.; Wang, L.; Ji, X.; Chen, Y.; Zheng, T.; Yu, J.; Feng, H. Graphene-Based Flexible Sensors for Simultaneous Detection of Ascorbic Acid, Dopamine, and Uric Acid. Front. Bioeng. Biotechnol. 2021, 9, No. 726071.
(11) Castagnola, E.; Garg, R.; Rastogi, S. K.; Cohen-Karni, T.; Cui, X. T. 3D Fuzzy Graphene Microelectrode Array for Dopamine Sensing at Sub-Cellular Spatial Resolution. Biosens. Bioelectron. 2021, 191, No. 113440.
(12) Zestos, A. G.; Venton, B. J. Communication—Carbon Nanotube Fiber Microelectrodes for High Temporal Measurements of Dopamine. J. Electrochem. Soc. 2018, 165 (12), G3071-G3073.
(13) Thielen, B.; Meng, E. A Comparison of Insertion Methods for Surgical Placement of Penetrating Neural Interfaces. J. Neural Eng. 2021, 18 (4), No. 041003.
(14) Riaz, M. A.; Chen, Y. Electrodes and Electrocatalysts for Electrochemical Hydrogen Peroxide Sensors: A Review of Design Strategies. Nanoscale Horiz. 2022, 7 (5), 463-479.
(15) Green, R.; Abidian, M. R. Conducting Polymers for Neural Prosthetic and Neural Interface Applications. Adv. Mater. 2015, 27 (46), 7620-7637.
(16) VahidMohammadi, A.; Rosen, J.; Gogotsi, Y. The World of Two-Dimensional Carbides and Nitrides (MXenes). Science 2021, 372 (6547), No. cabf1581.
(17) Abdolhosseinzadeh, S.; Jiang, X.; Zhang, H.; Qiu, J.; Zhang, C. John. Perspectives on Solution Processing of Two-Dimensional MXenes. Mater. Today 2021, 48, 214-240.
(18) Orts Mercadillo, V.; Chan, K. C.; Caironi, M.; Athanassiou, A.; Kinloch, I. A.; Bissett, M.; Cataldi, P. Electrically Conductive 2D Material Coatings for Flexible and Stretchable Electronics: A Comparative Review of Graphenes and MXenes. Adv. Funct. Mater. 2022, 32 (38), No. 2204772.
(19) Moscatelli, A. The Pull of the MXene Vortex. Nat. Nanotechnol. 2022, 17 (10), 1025.
(20) Shuck, C. E.; Sarycheva, A.; Anayee, M.; Levitt, A.; Zhu, Y.; Uzun, S.; Balitskiy, V.; Zahorodna, V.; Gogotsi, O.; Gogotsi, Y. Scalable Synthesis of Ti₃C₂T. MXene. Adv. Eng. Mater. 2020, 22 (3), No. 1901241.
(21) Georgantas, Y.; Moissinac, F. P.; Bissett, M. MXenes-Mining: A Decade of Discovery. Graphene 2D Mater. 2024, 9, 5-26.
(22) Garg, R.; Vitale, F. Latest Advances on MXenes in Biomedical Research and Health Care. MRS Bull. 2023, 48, 283-290.
(23) Aslam, M.; Ahmad, T.; Manzoor, M. H.; Laiba; Verpoort, F. MXenes as Theranostics: Diagnosis and Therapy Including in Vitro and in Vivo Applications. Appl. Mater. Today 2023, 35, No. 102002.
(24) Koyappayil, A.; Chavan, S. G.; Roh, Y.-G.; Lee, M.-H. Advances of MXenes; Perspectives on Biomedical Research. Biosensors 2022, 12 (7), 454.
(25) Wu, J.; Yu, Y.; Su, G. Safety Assessment of 2D MXenes: In Vitro and In Vivo. Nanomaterials 2022, 12 (5), 828.
(26) Wang, Y.; Garg, R.; Hartung, J. E.; Goad, A.; Patel, D. A.; Vitale, F.; Gold, M. S.; Gogotsi, Y.; Cohen-Karni, T. Ti₃C₂Tₓ MXene Flakes for Optical Control of Neuronal Electrical Activity. ACS Nano 2021, 15 (9), 14662-14671.
(27) Driscoll, N.; Richardson, A. G.; Maleski, K.; Anasori, B.; Adewole, O.; Lelyukh, P.; Escobedo, L.; Cullen, D. K.; Lucas, T. H.; Gogotsi, Y.; Vitale, F. Two-Dimensional Ti₃C₂ MXene for High-Resolution Neural Interfaces. ACS Nano 2018, 12 (10), 10419-10429.
(28) Xuc, Q.; Zhang, H.; Zhu, M.; Pei, Z.; Li, H.; Wang, Z.; Huang, Y.; Huang, Y.; Deng, Q.; Zhou, J.; Du, S.; Huang, Q.; Zhi, C. Photoluminescent Ti₃C₂ MXene Quantum Dots for Multicolor Cellular Imaging. Adv. Mater. 2017, 29 (15), 1604847.
(29) Dai, C.; Lin, H.; Xu, G.; Liu, Z.; Wu, R.; Chen, Y. Biocompatible 2D Titanium Carbide (MXenes) Composite Nanosheets for pH-Responsive MRI-Guided Tumor Hyperthermia. Chem. Mater. 2017, 29 (20), 8637-8652.
(30) Murphy, B. B.; Mulcahey, P. J.; Driscoll, N.; Richardson, A. G.; Robbins, G. T.; Apollo, N. V.; Maleski, K.; Lucas, T. H.; Gogotsi, Y.; Dillingham, T.; Vitale, F. A Gel-Frec Ti₃C₂Tₓ-Based Electrode Array for High-Density, High-Resolution Surface Electromyography. Adv. Mater. Technol. 2020, 5 (8), No. 2000325.
(31) Driscoll, N.; Erickson, B.; Murphy, B. B.; Richardson, A. G.; Robbins, G.; Apollo, N. V.; Mentzelopoulos, G.; Mathis, T.; Hantanasirisakul, K.; Bagga, P.; Gullbrand, S. E.; Sergison, M.; Reddy, R.; Wolf, J. A.; Chen, H. I.; Lucas, T. H.; Dillingham, T. R.; Davis, K. A.; Gogotsi, Y.; Medaglia, J. D.; Vitale, F. MXene-Infused Bioelectronic Interfaces for Multiscale Electrophysiology and Stimulation. Sci. Transl. Med. 2021, 13 (612), No. eabf8629.
(32) Garg, R.; Driscoll, N.; Shankar, S.; Hullfish, T.; Anselmino, E.; Iberite, F.; Averbeck, S.; Rana, M.; Micera, S.; Baxter, J. R.; Vitale, F. Wearable High-Density MXene-Bioelectronics for Neuromuscular Diagnostics, Rehabilitation, and Assistive Technologies. Small Methods 2023, 7, No. 2201318.
(33) Averbeck, S. R.; Xu, D.; Murphy, B. B.; Shevchuk, K.; Shankar, S.; Anayee, M.; Der Torossian Torres, M.; Beauchamp, M. S.; De La Fuente-Nunez, C.; Gogotsi, Y.; Vitale, F. Stability of Ti₃C₂Tₓ MXene Films and Devices under Clinical Sterilization Processes. ACS Nano 2023, 17 (10), 9442-9454.
(34) Lorencova, L.; Bertok, T.; Dosekova, E.; Holazova, A.; Paprckova, D.; Vikartovska, A.; Sasinkova, V.; Filip, J.; Kasak, P.; Jerigova, M.; Velic, D.; Mahmoud, K. A.; Tkac, J. Electrochemical Performance of Ti₃C₂Tₓ MXene in Aqueous Media: Towards Ultrasensitive H₂O₂ Sensing. Electrochim. Acta 2017, 235, 471-479.
(35) Sies, H. Hydrogen Peroxide as a Central Redox Signaling Molecule in Physiological Oxidative Stress: Oxidative Eustress. Redox Biol. 2017, 11, 613-619.
(36) Lee, M.; Shim, H. J.; Choi, C.; Kim, D.-H. Soft High-Resolution Neural Interfacing Probes: Materials and Design Approaches. Nano Lett. 2019, 19 (5), 2741-2749.
(37) Hejazi, M.; Tong, W.; Ibbotson, M. R.; Prawer, S.; Garrett, D. J. Advances in Carbon-Based Microfiber Electrodes for Neural Interfacing. Front. Neurosci. 2021, 15, No. 658703.
(38) Jiang, S.; Patel, D. C.; Kim, J.; Yang, S.; Mills, W. A.; Zhang, Y.; Wang, K.; Feng, Z.; Vijayan, S.; Cai, W.; Wang, A.; Guo, Y.; Kimbrough, I. F.; Sontheimer, H.; Jia, X. Spatially Expandable Fiber-Based Probes as a Multifunctional Deep Brain Interface. Nat. Commun. 2020, 11 (1), No. 6115.
(39) Gonzalez-Gonzalez, M. A.; Bendale, G. S.; Wang, K.; Wallace, G. G.; Romero-Ortega, M. Platinized Graphene Fiber Electrodes Uncover Direct Spleen-Vagus Communication. Commun. Biol. 2021, 4 (1), 1097.
(40) Falcone, J. D; Liu, T.; Goldman, L.; Pogue, D. D.; Rieth, L.; Bouton, C. E; Straka, M.; Sohal, H. S A Novel Microwire Interface for Small Diameter Peripheral Nerves in a Chronic, Awake Murine Model. J. Neural Eng. 2020, 17 (4), 046003.
(41) Loke, G.; Yan, W.; Khudiyev, T.; Noel, G.; Fink, Y. Recent Progress and Perspectives of Thermally Drawn Multimaterial Fiber Electronics. Adv. Mater. 2020, 32 (1), No. 1904911.
(42) Sahasrabudhe, A.; Rupprecht, L. E.; Orguc, S.; Khudiyev, T.; Tanaka, T.; Sands, J.; Zhu, W.; Tabet, A.; Manthey, M.; Allen, H.; Loke, G.; Antonini, M.-J.; Rosenfeld, D.; Park, J.; Garwood, I. C.; Yan, W.; Niroui, F.; Fink, Y.; Chandrakasan, A.; Bohorquez, D. V.; Anikeeva, P. Multifunctional Microelectronic Fibers Enable Wireless Modulation of Gut and Brain Neural Circuits. Nat. Biotechnol. 2024, 42, 892-904.
(43) Levitt, A.; Hegh, D.; Phillips, P.; Uzun, S.; Anayee, M.; Razal, J. M.; Gogotsi, Y.; Dion, G. 3D Knitted Energy Storage Textiles Using MXene-Coated Yarns. Mater. Today 2020, 34, 17-29.
(44) Uzun, S.; Seyedin, S.; Stoltzfus, A. L.; Levitt, A. S.; Alhabeb, M.; Anayee, M.; Strobel, C. J.; Razal, J. M.; Dion, G.; Gogotsi, Y. Knittable and Washable Multifunctional MXene-Coated Cellulose Yarns. Adv. Funct. Mater. 2019, 29 (45), No. 1905015.
(45) Kadolph, S. J.; Marcketti, S. B. Textiles; Pearson, 2017.
(46) Risbud, M. V.; Bhonde, R. R. Polyamide 6 Composite Membranes: Properties and in Vitro Biocompatibility Evaluation. J. Biomater. Sci., Polym. Ed. 2001, 12 (1), 125-136.
(47) Shakiba, M.; Rezvani Ghomi, E.; Khosravi, F.; Jouybar, S.; Bigham, A.; Zare, M.; Abdouss, M.; Moaref, R.; Ramakrishna, S. Nylon—A Material Introduction and Overview for Biomedical Applications. Polym. Adv. Technol. 2021, 32 (9), 3368-3383.
(48) Shekhirev, M.; Shuck, C. E.; Sarycheva, A.; Gogotsi, Y. Characterization of MXenes at Every Step, from Their Precursors to Single Flakes and Assembled Films. Prog. Mater. Sci. 2021, 120, No. 100757.
(49) MEMS Materials and Processes Handbook; Ghodssi, R.; Lin, P., Eds.; Springer Science & Business Media: Boston, MA, 2011; Vol. 1.
(50) Hassler, C.; Boretius, T.; Stieglitz, T. Polymers for Neural Implants: Polymers for Neural Implants. J. Polym. Sci., Part B: Polym. Phys. 2011, 49 (1), 18-33.
(51) Antonini, M.; Sahasrabudhe, A.; Tabet, A.; Schwalm, M.; Rosenfeld, D.; Garwood, I.; Park, J.; Loke, G.; Khudiyev, T.; Kanik, M.; Corbin, N.; Canales, A.; Jasanoff, A.; Fink, Y.; Anikeeva, P. Customizing MRI-Compatible Multifunctional Neural Interfaces through Fiber Drawing. Adv. Funct. Mater. 2021, 31 (43), No. 2104857.
(52) Park, S.; Guo, Y.; Jia, X.; Choe, H. K.; Grena, B.; Kang, J.; Park, J.; Lu, C.; Canales, A.; Chen, R.; Yim, Y. S.; Choi, G. B.; Fink, Y.; Anikeeva, P. One-Step Optogenetics with Multifunctional Flexible Polymer Fibers. Nat. Neurosci. 2017, 20 (4), 612-619.
(53) Bi, L.; Perry, W.; Wang, R.; Lord, R.; Hryhorchuk, T.; Inman, A.; Gogotsi, O.; Balitskiy, V.; Zahorodna, V.; Baginskiy, I.; Vorotilo, S.; Gogotsi, Y. MXene Functionalized Kevlar Yarn via Automated, Continuous Dip Coating. Adv. Funct. Mater. 2024, 34 (14), No. 2312434.
(54) Zhang, Z.; Salamatin, A.; Peng, F.; Kornev, K. G. Dip Coating of Cylinders with Newtonian Fluids. J. Colloid Interface Sci. 2022, 607, 502-513.
(55) Greaves, M.; Mende, M.; Wang, J.; Yang, W.; Barg, S. Investigating the Rheology of 2D Titanium Carbide (MXene) Dispersions for Colloidal Processing: Progress and Challenges. J. Mater. Res. 2021, 36 (22), 4578-4600.
(56) Woods, M. B.; Beidaghi, M.; Davis, V. A. Phase Behavior and Rheological Properties of Size-Fractionated MXene (Ti₃C₂Tₓ) Dispersions. Langmuir 2024, 40 (6), 2907-2917.
(57) Zhang, J.; Uzun, S.; Seyedin, S.; Lynch, P. A.; Akuzum, B.; Wang, Z.; Qin, S.; Alhabeb, M.; Shuck, C. E.; Lei, W.; Kumbur, E. C.; Yang, W.; Wang, X.; Dion, G.; Razal, J. M.; Gogotsi, Y. Additive-Free MXene Liquid Crystals and Fibers. ACS Cent. Sci. 2020, 6 (2), 254-265.
(58) Zhang, J.; Kong, N.; Uzun, S.; Levitt, A.; Seyedin, S.; Lynch, P. A.; Qin, S.; Han, M.; Yang, W.; Liu, J.; Wang, X.; Gogotsi, Y.; Razal, J. M. Scalable Manufacturing of Free-Standing, Strong Ti₃C₂Tₓ MXene Films with Outstanding Conductivity. Adv. Mater. 2020, 32 (3), No. 2001093.
(59) Mayer, H. C.; Krechetnikov, R. Landau-Levich Flow Visualization: Revealing the Flow Topology Responsible for the Film Thickening Phenomena. Phys. Fluids 2012, 24 (5), No. 052103.
(60) An, H.; Habib, T.; Shah, S.; Gao, H.; Patel, A.; Echols, I.; Zhao, X.; Radovic, M.; Green, M. J.; Lutkenhaus, J. L. Water Sorption in MXene/Polyelectrolyte Multilayers for Ultrafast Humidity Sensing. ACS Appl. Nano Mater. 2019, 2 (2), 948-955.
(61) An, H.; Habib, T.; Shah, S.; Gao, H.; Radovic, M.; Green, M. J.; Lutkenhaus, J. L. Surface-Agnostic Highly Stretchable and Bendable Conductive MXene Multilayers. Sci. Adv. 2018, 4 (3), No. eaaq0118.
(62) Tian, W.; VahidMohammadi, A.; Wang, Z.; Ouyang, L.; Beidaghi, M.; Hamedi, M. M. Layer-by-Layer Self-Assembly of Pillared Two-Dimensional Multilayers. Nat. Commun. 2019, 10 (1), No. 2558.
(63) Yun, J.; Echols, I.; Flouda, P.; Chen, Y.; Wang, S.; Zhao, X.; Holta, D.; Radovic, M.; Green, M. J.; Naraghi, M.; Lutkenhaus, J. L. Layer-by-Layer Assembly of Reduced Graphene Oxide and MXene Nanosheets for Wire-Shaped Flexible Supercapacitors. ACS Appl. Mater. Interfaces 2021, 13 (12), 14068-14076.
(64) Zhou, T.; Cao, C.; Yuan, S.; Wang, Z.; Zhu, Q.; Zhang, H.; Yan, J.; Liu, F.; Xiong, T.; Cheng, Q.; Wei, L. Interlocking-Governed Ultra-Strong and Highly Conductive MXene Fibers Through Fluidics-Assisted Thermal Drawing. Adv. Mater. 2023, 35 (51), No. 2305807.
(65) Wang, K.; Frewin, C. L.; Esrafilzadeh, D.; Yu, C.; Wang, C.; Pancrazio, J. J.; Romero-Ortega, M.; Jalili, R.; Wallace, G. High-Performance Graphene-Fiber-Based Neural Recording Microelectrodes. Adv. Mater. 2019, 31 (15), No. 1805867.
(66) Apollo, N. V.; Maturana, M. I.; Tong, W.; Nayagam, D. A. X.; Shivdasani, M. N.; Foroughi, J.; Wallace, G. G.; Prawer, S.; Ibbotson, M. R.; Garrett, D. J. Soft, Flexible Freestanding Neural Stimulation and Recording Electrodes Fabricated from Reduced Graphene Oxide. Adv. Funct. Mater. 2015, 25 (23), 3551-3559.
(67) Ni, Y.; Qi, H.; Zhang, F.; Jiang, S.; Tang, Q.; Cai, W.; Mo, W.; Miron, R. J.; Zhang, Y. Macrophages Modulate Stiffness-Related Foreign Body Responses through Plasma Membrane Deformation. Proc. Natl. Acad. Sci. U.S.A. 2023, 120 (3), No. e2213837120.
(68) Garwood, I. C.; Major, A. J.; Antonini, M.-J.; Correa, J.; Lee, Y.; Sahasrabudhe, A.; Mahnke, M. K.; Miller, E. K.; Brown, E. N.; Anikeeva, P. Multifunctional Fibers Enable Modulation of Cortical and Deep Brain Activity during Cognitive Behavior in Macaques. Sci. Adv. 2023, 9 (9), eadh0974.
(69) Krauss, J. K.; Lipsman, N.; Aziz, T.; Boutet, A.; Brown, P.; Chang, J. W.; Davidson, B.; Grill, W. M.; Hariz, M. I.; Horn, A.; Schulder, M.; Mammis, A.; Tass, P. A.; Volkmann, J.; Lozano, A. M. Technology of Deep Brain Stimulation: Current Status and Future Directions. Nat. Rev. Neurol. 2021, 17 (2), 75-87.
(70) Johnson, M. Transcutaneous Electrical Nerve Stimulation: Mechanisms, Clinical Application and Evidence. Rev. Pain 2007, 1 (1), 7-11.
(71) Noriega, N.; Shekhirev, M.; Shuck, C. E.; Salvage, J.; VahidMohammadi, A.; Dymond, M. K.; Lacey, J.; Sandeman, S.; Gogotsi, Y.; Patel, B. A. Pristine Ti₃C₂Tₓ MXene Enables Flexible and Transparent Electrochemical Sensors. ACS Appl. Mater. Interfaces 2024, 16 (5), 6569-6578.
(72) Cogan, S. F. Neural Stimulation and Recording Electrodes. Annu. Rev. Biomed. Eng. 2008, 10 (1), 275-309.
(73) Lee, A.; Shekhirev, M.; Anayee, M.; Gogotsi, Y. Multi-Year Study of Environmental Stability of Ti₃C₂Tₓ MXene Films. Graphene 2D Mater. 2024, 9, 77-85.
(74) Ahmad, S. I. Reactive Oxygen Species in Biology and Human Health; CRC Press, 2017.
(75) Lyons, M. K. Deep Brain Stimulation: Current and Future Clinical Applications. Mayo Clin. Proc. 2011, 86 (7), 662-672.
(76) Sluka, K. A.; Walsh, D. Transcutaneous Electrical Nerve Stimulation: Basic Science Mechanisms and Clinical Effectiveness. J. Pain 2003, 4 (3), 109-121.
(77) Feng, W.; Han, X.; Hu, H.; Chang, M.; Ding, L.; Xiang, H.; Chen, Y.; Li, Y. 2D Vanadium Carbide MXenzyme to Alleviate ROS-Mediated Inflammatory and Neurodegenerative Diseases. Nat. Commun. 2021, 12 (1), No. 2203.
(78) Shi, X.; Zuo, Y.; Zhai, P.; Shen, J.; Yang, Y.; Gao, Z.; Liao, M.; Wu, J.; Wang, J.; Xu, X.; Tong, Q.; Zhang, B.; Wang, B.; Sun, X.; Zhang, L.; Pei, Q.; Jin, D.; Chen, P.; Peng, H. Large-Area Display Textiles Integrated with Functional Systems. Nature 2021, 591 (7849), 240-245.
(79) Evertz, A.; Schrein, D.; Olsen, E.; Hoffmann, G.-A.; Overmeyer, L. Dip Coating of Thin Polymer Optical Fibers. Opt. Fiber Technol. 2021, 66, No. 102638.
(80) Lu, L.; Fu, X.; Licw, Y.; Zhang, Y.; Zhao, S.; Xu, Z.; Zhao, J.; Li, D.; Li, Q.; Stanley, G. B.; Duan, X. Soft and MRI Compatible Neural Electrodes from Carbon Nanotube Fibers. Nano Lett. 2019, 19 (3), 1577-1586.
(81) Overview of Materials for Nylon 66/6. https://www.matweb.com/search/datasheet_print.aspx?matguid=26386631ec1b49ceba62c80a 49730dc4. (accessed Jul. 29, 2023).
(82) MuGuinnesss, M. Parylene C Datasheet. https://www.hzo.com/blog/parylene-c-datasheet-pdf-download/. (accessed Jul. 29, 2023).

### Aspects

The following Aspects are illustrative only and do not limit the scope of the present disclosure or the appended claims. Any part or parts of any one or more Aspects can be combined with any part or parts of any one or more other Aspects.
Aspect 1. An electrode, comprising: a portion of MXene, the portion of MXene optionally being free of materials other than the MXene; and (a) a sealing material, the sealing material at least partially enclosing the portion of MXene, and the sealing material defining an opening through which a sensing region of the portion of MXene is exposed; or (b) a substrate, the portion of MXene being disposed on the substrate.

In certain embodiments, the MXene composition is any of the compositions described in at least one of U.S. patent application Ser. No. 17/767,083 (filed Oct. 9, 2020, Ser. No. 14/094,966 (filed Dec. 3, 2013), 62/055,155 (filed Sep. 25, 2014), 62/214,380 (filed Sep. 4, 2015), 62/149,890 (filed Apr. 20, 2015), 62/127,907 (filed Mar. 4, 2015) or International Applications PCT/US2012/043273 (filed Jun. 20, 2012), PCT/US2013/072733 (filed Dec. 3, 2013), PCT/US2015/051588 (filed Sep. 23, 2015), PCT/US2016/020216 (filed Mar. 1, 2016), or PCT/US2016/028,354 (filed Apr. 20, 2016), preferably where the MXene composition comprises titanium and carbon (e.g., Ti₃C₂, Ti₂C, Mo₂TiC₂, etc.)
Aspect 2. The electrode of Aspect 1, wherein the MXene is in electrical communication with a monitor configured to collect a signal from the MXene.
Aspect 3. The electrode of Aspect 2, wherein the signal relates to the exposure of the MXene to an analyte.
Aspect 4. The electrode of any one of Aspects 1-3, wherein the sensor component is comprised in a sensor system.
Aspect 5. The electrode of Aspect 4, wherein the sensor system is configured as an electrochemical sensor.
Aspect 6. The electrode of any one of Aspects 1-5, wherein the electrode is configured as a basal plane electrode.
Aspect 7. The electrode of any one of Aspects 1-5, wherein the electrode is configured as an edge plane electrode.
Aspect 8. The electrode of any one of Aspects 1-7, wherein the portion of MXene is essentially free of materials other than the MXene.
Aspect 9. The electrode of any one of Aspects 1-8, wherein the electrode is characterized as transparent.
Aspect 10. The electrode of any one of Aspects 1-9, wherein the electrode is flexible.
Aspect 11. The electrode of any one of Aspects 1-10, wherein the sealing material is a polymer.
Aspect 12. The electrode of any one of Aspects 1-11, wherein the MXene portion consists of MXene.
Aspect 13. A method, comprising use of an electrode according to any one of Aspects 1-12.
Aspect 14 A method, comprising contacting an electrode according to any one of Aspects 1-12 to an analyte and collecting a signal from the electrode related to the analyte.
Aspect 15. A method, comprising fabricating an electrode according to any one of Aspects 1-12.
Aspect 16. An electrode, comprising: a fiber; a first coating superposed on the fiber, the first coating having a thickness and comprising a MXene composition, the MXene composition optionally comprising aligned MXene flakes, the first coating optionally having a uniform thickness along the length of the fiber; and a second coating superposed on the fiber. It should be understood, however, that the second coating can be optional in some embodiments; in such embodiments, the electrode can comprise a fiber; a first coating superposed on the fiber, the first coating having a thickness and comprising a MXene composition, the MXene composition optionally comprising aligned MXene flakes, the first coating optionally having a uniform thickness along the length of the fiber, and the electrode being free of a second coating.

An example of such an electrode is provided in FIG. 10. As shown in that figure, an exemplary nylon fiber is first coated with MXene—T₃C₂Tₓ, in this instance—and a second coating—parylene, in this instance—is applied. One or both of the first coating and the second coating can be applied by, for example, dip coating. The first coating can have a thickness in the range of, for example, from about 10 nm to about 5 μm, or from about 100 nm to about 2.5 μm, and all intermediate values and sub-ranges. The second coating can have a thickness in the range of from, for example, about 1 μm to about 100 μm, about 5 μm to about 50 μm, or even from about 10 to about 20 μm.

Without being bound to any particular theory or embodiment, a fiber having the first coating superposed thereon can have a resistance in the range of from about 10 to about 200 Ω/cm, such as from about 10 to about 200 Ω/cm, from about 20 to about 100 Ω/cm, from about 30 to about 75 Ω/cm, from about 8 to about 20 Ω/cm, or even about 50 Ω/cm. A fiber having the first coating superposed thereon can have an impedance of from about 3 to about 20 kΩ at 1 KHz, for example from about 5 to about 15 kΩ at 1 kHz, or even from about 7 to about 12 kΩ at 1 KHz.

In some embodiments, the electrode can be free of a current collector, although this is not a requirement. In some embodiments, an electrode according to the present disclosure can be free of a current collector and be connected to a receiver, for example, a receiver that collects a signal from the electrode. Likewise, an electrode according to the present disclosure can be free of a current collector and be connected to a transmitter, for example, a voltage or current source that provides a voltage or current to the electrode.

It should be understood that although certain figures herein show the electrodes as individual electrodes, the disclosed electrodes can be present as multiple fibers, for example as a bundle of electrodes. Electrodes according to the present disclosure can be integrated into a textile, for example by way of weaving. A textile can also comprise the disclosed electrodes arranged in a nonwoven manner.

As described elsewhere herein, the fiber can comprise a charge—such as positive charge—that is opposite to a charge on the MXene. In this way, one can construct electrodes in which the MXene is associated with the fiber via positive-negative charge interactions. The functional groups on MXenes serve as effective anchoring points for binding other materials, such as antibodies and drugs. In this way, an electrode according to the present disclosure can be used in a drug delivery application.

As shown in FIG. 10 (a), the first coating can include aligned MXene flakes. Without being bound to any particular theory or embodiment, MXene flakes can be aligned via a shear-assisted approach, which alignment can take place in connection with a dip coating or other coating process.

It should be understood that the fiber need not necessarily comprise nylon, as other fiber materials-such as polypropylene, polyethylene—are also suitable. Similarly, the MXene coating need not necessarily comprise T₃C₂Tₓ, as other MXenes can also be used. Likewise, the second coating need not necessarily comprise parylene, as other coating materials—such as other polymers—can be used.
Aspect 17. The electrode of Aspect 16, wherein the fiber comprises a polymer, the polymer optionally comprising a polyamide.
Aspect 18. The electrode of any one of Aspects 16-17, wherein the fiber defines a diameter of from about 10 to about 1000 μm, optionally from about 10 to about 500 μm. A fiber can have a diameter of, for example from about 10 to about 1000 μm, from about 50 to about 750 μm, from about 100 to about 500 μm, and all intermediate values and sub-ranges.
Aspect 19. The electrode of any one of Aspects 16-18, wherein the second coating is disposed on the first coating.
Aspect 20. The electrode of any one of Aspects 16-19, wherein the second coating comprises a polymer, the polymer optionally comprising parylene. Other polymers besides parylene can be used.
Aspect 21. The electrode of any one of Aspects 16-20, wherein the electrode is in electrical communication with a monitor configured to collect a signal from the portion of MXene. Such a signal can, for example, be an electrical signal.
Aspect 22. A method, comprising contacting an electrode according to any one of Aspects 16-21 to a tissue, for example tissue of a subject. Such contacting can be, for example, contacting the end of the electrode to the subject's tissue. Such tissue can be, as but some examples, brain tissue, muscle tissue, organoids, or even cells. It should also be understood that tissue need not necessary be associated with a subject. As but one example, electrodes according to the present disclosure can be contacted to synthetic, ex vivo, or in vitro tissues, such as organoids. Likewise, electrodes according to the present disclosure can be contacted to cells. Such cells can be cells of a subject, but this is not a requirement, as cells can also be in culture or otherwise in vitro.
Aspect 23. A method, comprising inserting an electrode according to any one of Aspects 16-21 into a subject. The insertion can be, for example, into brain tissue, bladder tissue, muscular tissue, and the like. Without being bound to any particular theory, the electrode can be of sufficient stiffness that the electrode can be inserted directly into the tissue of interest.
Aspect 24. A method, comprising collecting a signal with an electrode according to any one of Aspects 16-21, the signal optionally relating to the evolution of a product. As but one example, the signal can be related to the production of hydrogen peroxide.
Aspect 25. A method, comprising at least one of recording and stimulating physiologic activity with an electrode according to any one of Aspects 16-21. The recording can be, for example, of neurological activity, muscular activity, cardiac activity, and the like. Similarly, the stimulating can be, for example, of neurological activity, muscular activity, cardiac activity, and the like. A user can use electrodes according to the present disclosure to stimulate and record activity; the electrodes can be arranged such that one electrode according to the present disclosure is a recording electrode, and another electrode according to the present disclosure can be a stimulating electrode.
Aspect 26. A method, comprising removing a portion of an electrode according to any one of Aspects 16-21 so as to expose a cross-section of the fiber, the first coating, and the second coating. This can be performed so that a given electrode can be used in multiple experiments.

For example, in a first experiment, the face of the end of the electrode can be exposed to a sample of interest. Following the completion of that first experiment, the user can cut off the end of the electrode, thereby exposing a fresh face of the electrode, which fresh face can then be used in a second experiment. In this way, a single electrode according to the present disclosure can be used in multiple experiments simply by trimming off the end of the electrode before each successive experiment. Such a configuration hence saves material and time, as a user does not need to use a new electrode for each experiment the user may perform. As shown in FIG. 10(c), after an encapsulated fiber is cut at the tip, a controllable amount of MXene can be exposed, providing the benefits of easy handling and reproducible outcomes. An electrode according to the present disclosure can be arranged such that the end face of the electrode is flat and perpendicular to the major axis of the fiber. But this is not a requirement, as the end face can be angled, bevelled, curved, scooped, or otherwise non-perpendicular to the major axis of the fiber. Without being bound to any particular theory or embodiment, an angled end face can be easier to insert into a tissue.

What is claimed:

1. An electrode, comprising:
a portion of MXene, the portion of MXene optionally being free of materials other than the MXene; and
(a) a sealing material,
the sealing material at least partially enclosing the portion of MXene, and
the sealing material defining an opening through which a sensing region of the portion of MXene is exposed; or
(b) a substrate, the portion of MXene being disposed on the substrate.

2. The electrode of claim 1, wherein the portion of MXene is in electrical communication with a monitor configured to collect a signal from the portion of MXene.

3. The electrode of claim 2, wherein the signal relates to an exposure of the portion of MXene to an analyte.

4. The electrode of claim 1, wherein the electrode is comprised in a sensor system, the sensor system optionally configured as an electrochemical sensor.

5. The electrode of claim 1, wherein the electrode is configured as a basal plane electrode.

6. The electrode of claim 1, wherein the electrode is configured as an edge plane electrode.

7. The electrode of claim 1, wherein the electrode is characterized as transparent.

8. The electrode of claim 1, wherein the electrode is flexible.

9. The electrode of claim 1, wherein the sealing material is a polymer.

10. An electrode, comprising:
a fiber;
a first coating superposed on the fiber,
the first coating having a thickness and comprising a MXene composition,
the MXene composition optionally comprising aligned MXene flakes,
the first coating optionally having a uniform thickness along the length of the fiber; and
a second coating superposed on the fiber.

11. The electrode of claim 10, wherein the fiber comprises a polymer, the polymer optionally comprising a polyamide.

12. The electrode of claim 10, wherein the fiber defines a diameter of from about 10 to about 1000 μm, optionally from about 10 to about 500 μm.

13. The electrode of claim 10, wherein the second coating is disposed on the first coating.

14. The electrode of claim 10, wherein the second coating comprises a polymer, the polymer optionally comprising parylene.

15. The electrode of claim 10, wherein the electrode is in electrical communication with a monitor configured to collect a signal from the portion of MXene.

16. A method, comprising contacting an electrode according to claim 10 to a tissue, the tissue optionally being of a subject.

17. A method, comprising inserting an electrode according to claim 10 into a subject.

18. A method, comprising collecting a signal with an electrode according to claim 10, the signal optionally relating to the evolution of a product.

19. A method, comprising at least one of recording and stimulating physiologic activity with an electrode according to claim 10.

20. A method, comprising removing a portion of an electrode according to claim 10 so as to expose a cross-section of the fiber, the first coating, and the second coating.
